(19) United States
(12) Patent Application Publication
(10) Pub. No.: US 2021/0101905 A1
(43) Pub. Date: Apr. 8, 2021

(54) Development of Imaging and Therapeutic Glucose Analogues for Sodium Dependent Glucose Transporters

(71) Applicants: Llnited States Government as represented by the Department of Veterans Affairs, Washington, DC (US); Case Western Reserve University, Cleveland, OH (US)

(72) Inventors: Richard S. Agnes, Cleveland Heights, OH (US); Bryan J. Traughber, Hummelstown, PA (US); Raymond F. Muzic, JR., Mentor, OH (US)

(21) Appl. No.: 17/062,018

(22) Filed: Oct. 2, 2020

Related U.S. Application Data
(60) Provisional application No. 62/910,892, filed on Oct. 4, 2019.

(51) Int. Cl. C07D 491/107; C07D 491/22; C07H 23/00; C07B 59/00

(52) U.S. Cl. CPC C07D 491/107; C07D 491/22; C07B 2200/05; C07B 59/002; C07B 59/005; C07H 23/00

(57) ABSTRACT

The present disclosure describes glucose analogs that are transported on the sodium dependent glucose transporters (SGLTs). These compounds may be useful in a variety of disorders such as, for example, cancer, heart disease, neurological disorders, diabetes, and atherosclerosis. This abstract is intended as a scanning tool for purposes of searching in the particular art and is not intended to be limiting of the present invention.

### CROSS-REFERENCE TO RELATED APPLICATIONS

This application claims priority to U.S. Provisional Application Ser. No. 62/910,892, filed Oct. 4, 2019, the contents of which are incorporated herein by reference in their entirety.

### BACKGROUND

Prostate cancer (PCa) is the most frequently-diagnosed cancer among veterans. Further, exposure to Agent Orange is associated with particularly aggressive PCa: a 75% increased risk of high-grade PCa, two-fold increase in detecting PCa with a Gleason score of 8 or greater, a significant biochemical progression risk, and a shorter PSA doubling time after recurrence (8.2 vs 18.6 months). Unfortunately, after PCa has metastasized there is no cure. Initially, metastatic prostate cancer responds to androgen-deprivation therapy (ADT) but often becomes resistant (metastatic, castration-resistant prostate cancer, mCRPC) and death occurs within 1 to 4 years. Specifically, the 5-year survival is only 29%. Therefore, new therapeutic approaches are needed.

The reliance of many cancers on glucose for energy necessitates efficient transport mechanisms to bring this water-soluble molecule through the lipid bilayer plasma membrane. Indeed, glucose transporters (GLUTs) transport glucose into cells at rates 10,000-fold greater than by diffusion (Elbrink and Bihler (1975) Science 188:1177-84) and are key to bringing the clinically-used PET imaging agent ¹⁸F-labeled 2-fluoro-2-deoxy-D-glucose ([¹⁸F]FDG) inside the cell. Whereas the role of GLUTs in cancer has been long appreciated, the role of sodium-dependent glucose transporters (SGLTs) is only recently emerging. Further, SGLTs have tremendous promise as a new cancer biomarker and theranostic target; different from GLUTs, which only move glucose down its concentration gradient, SGLTs can concentrate glucose and its analogs intracellularly from 10- to 400-fold their extracellular concentration (Kimmich and Randles (1981) The American journal of physiology 241:C227-3). Further, SGLT1 can process 1000 molecules per second (Wright et al. (2004) Physiology (Bethesda) 19:370-6). These properties of SGLTs may be key to tumor growth, supporting proliferation in environments having much lower glucose concentrations than in corresponding normal tissues (Urasaki et al. (2012) PloS one 7:e36775). Indeed, the survival advantage due to SGLT expression in a low glucose environment has been demonstrated previously in prostate cancer cell systems: knocking down SGLT1 by siRNA causes autophagic cell death in a euglycemic concentration and cells can be rescued by increasing the glucose concentration five-fold (Weihua et al. (2008) Cancer cell 13:385-93. Further, in A549 human bronchial carcinoma cells challenged with ionizing radiation, SGLT1 supports a seven-fold increased glucose uptake whereas SGLT1 inhibition enhanced cell-killing caused by irradiation in both A549 and SAS squamous cell carcinoma tumor cell lines but not in control studies in HSF7 normal skin fibroblasts (Dittmann et al. (2013) Radiotherapy and oncology: journal of the European Society for Therapeutic Radiology and Oncology 107:247-51). In normal physiological states, SGLTs are conventionally recognized as being localized in the proximal tubule of the kidneys where they reabsorb glucose into the blood so that it is not excreted in urine; and also the intestinal mucosa where they help to absorb sugar. To date, SGLTs cannot be probed using clinical [¹⁸F]FDG-PET as [¹⁸F]FDG is poorly transported by SGLTs as evidenced by previous work demonstrating high urinary excretion both in the absence and presence of SGLT inhibitor (Landau et al. (2007) American journal of physiology Endocrinology and metabolism 293:E237-45; Kobayashi et al. (2010) Nuclear medicine communications 31:141-6). While ¹⁸F-labeled 6-fluoro-6-deoxy-D-glucose ([¹⁸F]6 FDG), a previously developed PET compound (Landau et al. (2007) American journal of physiology Endocrinology and metabolism 293:E237-45; Muzic et al. (2011) Nuclear medicine and biology 38:667-74; Neal et al. (2005) J Labelled Compd Rad 48:845-54; Spring-Robinson et al. (2009) Journal of nuclear medicine: official publication, Society of Nuclear Medicine 50:912-9; Huang et al. (2012) Physiological measurement 33:1661-73; Muzic et al. (2014) Medical physics 41:031910; Su et al. (2014) Molecular imaging and biology: MIB: the official publication of the Academy of Molecular Imaging 16:710-20), has potential for imaging SGLTs, it is less than ideal as it is non-selective being also transported by GLUTs. Thus, there is a need for an SGLT-selective agent and methods of making and using same.

### SUMMARY

In accordance with the purpose(s) of the invention, as embodied and broadly described herein, the invention, in one aspect, relates to compositions and methods for use in the modification of SGLT expression. Such compositions may be useful in a variety of diseases and disorders such as, for example, disorders of uncontrolled cellular proliferation, neurological disorders, atherosclerosiss, diabetes, and heart disease.

Disclosed are compounds having a structure represented by a formula:

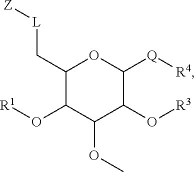

wherein L is a divalent linker; wherein Q is selected from —O—, —NHC(O)—, and —NHC(S)NH—; wherein Z is a fluorophore, a radiolabel, a radioisotope, or a radiotherapeutic; and wherein each of R¹, R², R³, and R⁴ is independently selected from hydrogen, C1-C4 alkyl, aryl, a fluorophore residue, a peptide residue, and a polyaromatic residue, provided that when Q is —O— and Z is a fluorophore, then at least one of R¹, R², R³, and R⁴ is not hydrogen, or a pharmaceutically acceptable salt thereof.

Also disclosed are pharmaceutical compositions comprising a therapeutically effective amount of a disclosed compound, and a pharmaceutically acceptable carrier.

Also disclosed are methods for the treatment of a disorder associated with SGLT expression in a subject, the method comprising administering to the subject an effective amount of at least one disclosed compound, wherein the disorder is a disorder of uncontrolled cellular proliferation, a neurological disorder, atherosclerosis, diabetes, or heart disease.

Also disclosed are methods for modifying sodium-dependent glucose transporter (SGLT) expression in a subject, the method comprising administering to the subject an effective amount of at least one disclosed compound.

Also disclosed are methods for modifying SGLT expression in at least one cell, the method comprising the step of contacting at least one cell with an effective amount of at least one disclosed compound.

Also disclosed are kits comprising at least one disclosed compound, and one or more of: (a) at least one agent associated with the treatment of a disorder associated with SGLT expression, wherein the disorder is selected from a disorder of uncontrolled cellular proliferation, a neurological disorder, atherosclerosis, diabetes, and heart disease; (b) instructions for administering the compound in connection with treating the disorder; and (c) instructions for treating the disorder.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### BRIEF DESCRIPTION OF THE DRAWINGS

The accompanying figures, which are incorporated in and constitute a part of this specification, illustrate several aspects and together with the description serve to explain the principles of the invention.

FIG. 1A shows representative structures of D-glucose analogues used for monitoring glucose cell uptake. For example, 2-fluoro-deoxy-D-glucose (2FDG, left) and 6-fluorodeoxy-D-glucose (6FDG, middle) are radioactive PET imaging tracers. 2-nitrobenzofurazan-D-glucose (2NBDG, right) is a fluorescent, non-radioactive agent used for assays for glucose transpot studies.

FIG. 1B shows a representative schematic illustrating the design of near-infrared fluorescent glucose analogues having a modification at C-6.

FIG. 2A-D show representative images illustrating the structure of 6FGA (FIG. 2A), the synthetic route to access 6FGA via “click” chemistry (FIG. 2B), an HPLC trace demonstating the purification of 6FGA (FIG. 2C), and characterization of 6FGA by mass spectrometry (FIG. 2D).

FIG. 3 shows a representative schematic illustrating “click” chemistry labeling with a clinical radionuclide, e.g., ⁶⁸Ga, ¹⁷⁷Lu, ⁸⁹Zr, among others.

FIG. 4 shows a representative PET image of a transgenic mouse model of cancer illustrating [¹⁸F]6FDG concentrated in a tumor. Subsequent genetic analysis revealed upregulated slc5A1 (SGLT1) mRNA.

FIG. 5A and FIG. 5B show representative Western blots demonstrating SGLTs in prostate PC-3 and breast MDA-MB-231 cancer cells (FIG. 5A) and SGLT2 is also expressed in LNCaP (FIG. 5B).

FIG. 6 shows representative data illustrating 6FGA transport into cancer cells. Specifically, uptake in MDA-MB-231 TNBC cells as control (panel A), and in the presence of GLUT inhibitor cytochalasin (panel B), but not with SGLT inhibitors phlorizin (panel C) and the highly SGLT2-selective dapagliflozin (panel D). Further confirming the SGLT mechanism, 6FGA uptake occurs when the co-transported sodium is in the media in MDA-MB-231 (panel E) and PC-3 (panel F) cells but not when sodium is absent from the media (panels G and H).

FIG. 7 shows representative images illustrating that the brain does not show fluorescence from 6FGA. Photographs show the location f tissue slices and the corresponding fluorescence images show 6FGA. No fluorescence is observed in the brain.

FIG. 8A and FIG. 8B show representative data illustrating that 6FGA is suitable for in vivo study. Referring to FIG. 8A, a MTT assay for low and high concentration of 6FGA in PC-3 for 24 hours showed that 6FGA is not cytotoxic to cells. Referring to FIG. 8B, ex vivo incubation of PC-3 tumor sections demonstrated increased uptake over time. Without wishing to be bound by theory, these data suggest that 6FGA can be used to explore SGLT targeting in mice.

FIG. 9 shows representative images illustrating that 6FGA concentrates in tumor overtime. 6FGA was injected i.v. into mice bearing PC-3 tumor in the flank. In vivo fluorescence of 6FGA was observed at 1, 16, and 24 hours using Maestro fluorescence imaging system. 6FGA concentrates in tumor mass at t=1 hour and increases over time. Without wishing to be bound theory, these suggest the potential of SGLTs as a therapeutic target.

FIG. 10 shows representative images illustrating high-resolution cryo-image of green fluorescent protein (GFP) murine metastasis model. Insets show brain (panel A), liver (panel B), lungs (panel C), and vertebra (panel D). The bars are 1 mm.

FIG. 11 shows representative data illustrating MIRDcell cell survival simulation assuming 50% of cells are labeled with ¹⁷⁷Lu. “Cross-fire” (radiation deposition in nearby cells that are not specifically targeted) is reflected by unlabeled cells tht are killed by radiation emanating from their labeled neighbors. See https://pubmed.ncbi.nlm.nih.gov/25012457/; see also J Nucl Med. 2014 September; 55(9):1557-64. doi: 10.2967/jnumed.113.131037. Epub 2014 Jul. 10. MIRD pamphlet No. 25: MIRDcell V2.0 software tool for dosimetric analysis of biologic response of multicellular populations.

Behrooz Vaziri, Han Wu, Atam P Dhawan, Peicheng Du, Roger W Howell, SNMMI MIRD Committee

FIG. 12 shows a representative generic structure of SGLT analogs.

FIG. 13 shows a representative schematic illustrating the synthesis of a radiolabeled 6-azido glucose analog via DOTA (tetraxetan) azide/alkyne.

FIG. 14 shows a representative schematic illustrating the synthesis of a radiolabeled 6-azido glucose analog via DOTA azide/DBCO (dibenzocyclooctyne).

FIG. 15 shows a representative schematic illustrating the synthesis of a radiolabeled 6-azido glucose analog via an amide.

FIG. 16 shows a representative schematic illustrating the synthesis of a radiolabeled 6-azido glucose analog via NOTA (1,4,7-Triazacyclononane-1,4,7-triacetic acid) SCN.

FIG. 17 shows representative schematics illustrating the synthesis of radiolabeled 6-azido-O-methyl-glucoside analogs.

FIG. 18 shows representative schematics illustrating the synthesis of radiolabeled 6-azido-O-amino-glucoside analogs, dual-labeled at the 1- and 6-positions.

FIG. 19 shows representative schematics illustrating non-chelate labeling of glucose analogs via click chemistry.

FIG. 20 shows a synthetic scheme for the synthesis of a radiolabeled glucose analogue targeting SGLT.

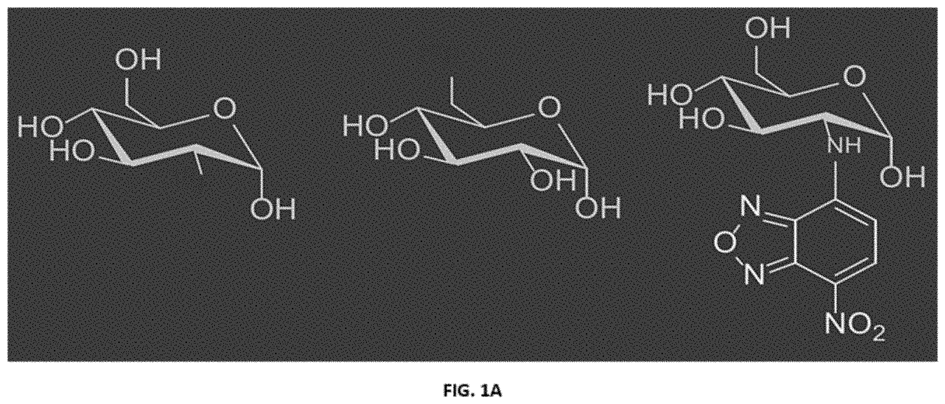

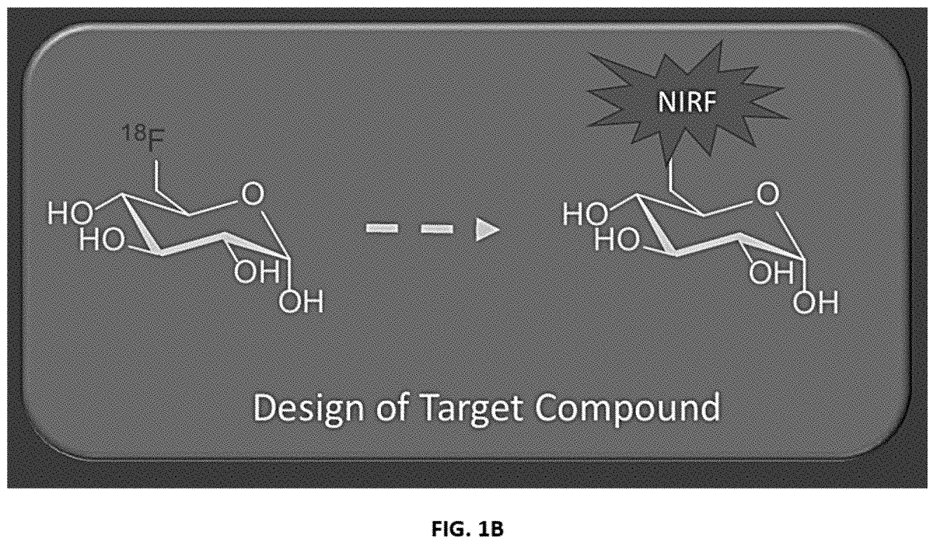

Additional advantages of the invention will be set forth in part in the description that follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such publication by virtue of prior invention. Further, the dates of publication provided herein may be different from the actual publication dates, which can require independent confirmation.

### A. Definitions

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

As used in the specification and in the claims, the term “comprising” can include the aspects “consisting of” and “consisting essentially of.”

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about” and “at or about” mean that the amount or value in question can be the value designated some other value approximately or about the same. It is generally understood, as used herein, that it is the nominal value indicated 10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the terms “optional” or “optionally” means that the subsequently described event or circumstance can or cannot occur, and that the description includes instances where said event or circumstance occurs and instances where it does not.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, “dosage form” means a pharmacologically active material in a medium, carrier, vehicle, or device suitable for administration to a subject. A dosage forms can comprise inventive a disclosed compound, a product of a disclosed method of making, or a salt, solvate, or polymorph thereof, in combination with a pharmaceutically acceptable excipient, such as a preservative, buffer, saline, or phosphate buffered saline. Dosage forms can be made using conventional pharmaceutical manufacturing and compounding techniques. Dosage forms can comprise inorganic or organic buffers (e.g., sodium or potassium salts of phosphate, carbonate, acetate, or citrate) and pH adjustment agents (e.g., hydrochloric acid, sodium or potassium hydroxide, salts of citrate or acetate, amino acids and their salts) antioxidants (e.g., ascorbic acid, alpha-tocopherol), surfactants (e.g., polysorbate 20, polysorbate 80, polyoxyethylene 9-10 nonyl phenol, sodium desoxycholate), solution and/or cryo/lyo stabilizers (e.g., sucrose, lactose, mannitol, trehalose), osmotic adjustment agents (e.g., salts or sugars), antibacterial agents (e.g., benzoic acid, phenol, gentamicin), antifoaming agents (e.g., polydimethylsilozone), preservatives (e.g., thimerosal, 2-phenoxyethanol, EDTA), polymeric stabilizers and viscosity-adjustment agents (e.g., polyvinylpyrrolidone, poloxamer 488, carboxymethylcellulose) and co-solvents (e.g., glycerol, polyethylene glycol, ethanol). A dosage form formulated for injectable use can have a disclosed compound, a product of a disclosed method of making, or a salt, solvate, or polymorph thereof, suspended in sterile saline solution for injection together with a preservative.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form, which may be supplied on computer readable memory device or downloaded from an internet website, or as recorded presentation. Instructions can comprise one or multiple documents, and are meant to include future updates.

As used herein, the terms “therapeutic agent” include any synthetic or naturally occurring biologically active compound or composition of matter which, when administered to an organism (human or nonhuman animal), induces a desired pharmacologic, immunogenic, and/or physiologic effect by local and/or systemic action. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14^{th} edition), the Physicians' Desk Reference (64^{th} edition), and The Pharmacological Basis of Therapeutics (12^{th} edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term “therapeutic agent” also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

The term “pharmaceutically acceptable” describes a material that is not biologically or otherwise undesirable, i.e., without causing an unacceptable level of undesirable biological effects or interacting in a deleterious manner.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, and amides, salts of esters or amides, and N-oxides of a parent compound.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions that can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” or “aliphatic group,” as used herein, denotes a hydrocarbon moiety that may be straight chain (i.e., unbranched), branched, or cyclic (including fused, bridging, and spirofused polycyclic) and may be completely saturated or may contain one or more units of unsaturation, but which is not aromatic. Unless otherwise specified, aliphatic groups contain 1-20 carbon atoms. Aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. Alternatively, the term “monohaloalkyl” specifically refers to an alkyl group that is substituted with a single halide, e.g. fluorine, chlorine, bromine, or iodine. The term “polyhaloalkyl” specifically refers to an alkyl group that is independently substituted with two or more halides, i.e. each halide substituent need not be the same halide as another halide substituent, nor do the multiple instances of a halide substituent need to be on the same carbon. The term “alkoxyalkyl” specifically refers to an alkyl group that is substituted with one or more alkoxy groups, as described below. The term “aminoalkyl” specifically refers to an alkyl group that is substituted with one or more amino groups. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether, halide, hydroxy, nitro, silyl, sulfo-oxo, or thiol as described herein.

The term “polyalkylene group” as used herein is a group having two or more CH₂ groups linked to one another. The polyalkylene group can be represented by the formula —(CH₂)ₐ—, where “a” is an integer of from 2 to 500.

The terms “alkoxy” and “alkoxyl” as used herein to refer to an alkyl or cycloalkyl group bonded through an ether linkage; that is, an “alkoxy” group can be defined as -OA¹ where A¹ is alkyl or cycloalkyl as defined above. “Alkoxy” also includes polymers of alkoxy groups as just described; that is, an alkoxy can be a polyether such as -OA¹-OA² or -OA¹-(OA²)ₐ-OA³, where “a” is an integer of from 1 to 200 and A, A², and A³ are alkyl and/or cycloalkyl groups.

The term “alkenyl” as used herein is a hydrocarbon group of from 2 to 24 carbon atoms with a structural formula containing at least one carbon-carbon double bond.

Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbomenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aromatic group” as used herein refers to a ring structure having cyclic clouds of delocalized π electrons above and below the plane of the molecule, where the π clouds contain (4n+2) π electrons. A further discussion of aromaticity is found in Morrison and Boyd, Organic Chemistry, (5th Ed., 1987), Chapter 13, entitled “Aromaticity,” pages 477-497, incorporated herein by reference. The term “aromatic group” is inclusive of both aryl and heteroaryl groups.

The term “aryl” as used herein is a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, —NH₂, carboxylic acid, ester, ether, halide, hydroxy, ketone, azide, nitro, silyl, sulfo-oxo, or thiol as described herein. The term “biaryl” is a specific type of aryl group and is included in the definition of “aryl.” In addition, the aryl group can be a single ring structure or comprise multiple ring structures that are either fused ring structures or attached via one or more bridging groups such as a carbon-carbon bond. For example, biaryl can be two aryl groups that are bound together via a fused ring structure, as in naphthalene, or are attached via one or more carbon-carbon bonds, as in biphenyl.

The term “aldehyde” as used herein is represented by the formula —C(O)H. Throughout this specification “C(O)” is a short hand notation for a carbonyl group, i.e., C═O.

The terms “amine” or “amino” as used herein are represented by the formula -NA¹A², where A¹ and A² can be, independently, hydrogen or alkyl, cycloalkyl, alkenyl, cycloalkenyl, alkynyl, cycloalkynyl, aryl, or heteroaryl group as described herein. A specific example of amino is —NH₂.

The term “alkylamino” as used herein is represented by the formula —NH(-alkyl) where alkyl is a described herein. Representative examples include, but are not limited to, methylamino group, ethylamino group, propylamino group, isopropylamino group, butylamino group, isobutylamino group, (sec-butyl)amino group, (tert-butyl)amino group, pentylamino group, isopentylamino group, (tert-pentyl)amino group, hexylamino group, and the like.

The term “dialkylamino” as used herein is represented by the formula —N(-alkyl)₂ where alkyl is a described herein. Representative examples include, but are not limited to, dimethylamino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylamino group, N-methyl-N-propylamino group, N-ethyl-N-propylamino group and the like.

The term “carboxylic acid” as used herein is represented by the formula —C(O)OH.

The term “ester” as used herein is represented by the formula —OC(O)A¹ or —C(O)OA¹, where A¹ can be alkyl, cycloalkyl, alkenyl, cycloalkenyl, alkynyl, cycloalkynyl, aryl, or heteroaryl group as described herein. The term “polyester” as used herein is represented by the formula -(A¹O(O)C-A²-C(O)O)ₐ— or -(A¹O(O)C-A²-OC(O))ₐ—, where A¹ and A² can be, independently, an alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “ether” as used herein is represented by the formula A¹OA², where A and A² can be, independently, an alkyl, cycloalkyl, alkenyl, cycloalkenyl, alkynyl, cycloalkynyl, aryl, or heteroaryl group described herein. The term “polyether” as used herein is represented by the formula -(A¹O-A²O)ₐ—, where A and A² can be, independently, an alkyl, cycloalkyl, alkenyl, cycloalkenyl, alkynyl, cycloalkynyl, aryl, or heteroaryl group described herein and “a” is an integer of from to 500. Examples of polyether groups include polyethylene oxide, polypropylene oxide, and polybutylene oxide.

The terms “halo,” “halogen,” or “halide,” as used herein can be used interchangeably and refer to F, Cl, Br, or I.

The terms “pseudohalide,” “pseudohalogen,” or “pseudohalo,” as used herein can be used interchangeably and refer to functional groups that behave substantially similar to halides. Such functional groups include, by way of example, cyano, thiocyanato, azido, trifluoromethyl, trifluoromethoxy, perfluoroalkyl, and perfluoroalkoxy groups.

The term “heteroalkyl,” as used herein refers to an alkyl group containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. Heteroalkyls can be substituted as defined above for alkyl groups.

The term “heteroaryl,” as used herein refers to an aromatic group that has at least one heteroatom incorporated within the ring of the aromatic group. Examples of heteroatoms include, but are not limited to, nitrogen, oxygen, sulfur, and phosphorus, where N-oxides, sulfur oxides, and dioxides are permissible heteroatom substitutions. The heteroaryl group can be substituted or unsubstituted. The heteroaryl group can be substituted with one or more groups including, but not limited to, alkyl, cycloalkyl, alkoxy, amino, ether, halide, hydroxy, nitro, silyl, sulfo-oxo, or thiol as described herein. Heteroaryl groups can be monocyclic, or alternatively fused ring systems. Heteroaryl groups include, but are not limited to, furyl, imidazolyl, pyrimidinyl, tetrazolyl, thienyl, pyridinyl, pyrrolyl, N-methylpyrrolyl, quinolinyl, isoquinolinyl, pyrazolyl, triazolyl, thiazolyl, oxazolyl, isoxazolyl, oxadiazolyl, thiadiazolyl, isothiazolyl, pyridazinyl, pyrazinyl, benzofuranyl, benzodioxolyl, benzothiophenyl, indolyl, indazolyl, benzimidazolyl, imidazopyridinyl, pyrazolopyridinyl, and pyrazolopyrimidinyl. Further not limiting examples of heteroaryl groups include, but are not limited to, pyridinyl, pyridazinyl, pyrimidinyl, pyrazinyl, thiophenyl, pyrazolyl, imidazolyl, benzo[d]oxazolyl, benzo[d]thiazolyl, quinolinyl, quinazolinyl, indazolyl, imidazo[1,2-b]pyridazinyl, imidazo[1,2-a]pyrazinyl, benzo[c][1,2,5]thiadiazolyl, benzo[c][1,2,5]oxadiazolyl, and pyrido[2,3-b]pyrazinyl.

The terms “heterocycle” or “heterocyclyl,” as used herein can be used interchangeably and refer to single and multi-cyclic aromatic or non-aromatic ring systems in which at least one of the ring members is other than carbon. Thus, the term is inclusive of, but not limited to, “heterocycloalkyl”, “heteroaryl”, “bicyclic heterocycle” and “polycyclic heterocycle.” Heterocycle includes pyridine, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridazine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like. The term heterocyclyl group can also be a C2 heterocyclyl, C2-C3 heterocyclyl, C2-C4 heterocyclyl, C2-C5 heterocyclyl, C2-C6 heterocyclyl, C2-C7 heterocyclyl, C2-C8 heterocyclyl, C2-C9 heterocyclyl, C2-C10 heterocyclyl, C2-C11 heterocyclyl, and the like up to and including a C2-C18 heterocyclyl. For example, a C2 heterocyclyl comprises a group which has two carbon atoms and at least one heteroatom, including, but not limited to, aziridinyl, diazetidinyl, dihydrodiazetyl, oxiranyl, thiiranyl, and the like. Alternatively, for example, a C5 heterocyclyl comprises a group which has five carbon atoms and at least one heteroatom, including, but not limited to, piperidinyl, tetrahydropyranyl, tetrahydrothiopyranyl, diazepanyl, pyridinyl, and the like. It is understood that a heterocyclyl group may be bound either through a heteroatom in the ring, where chemically possible, or one of carbons comprising the heterocyclyl ring.

The term “bicyclic heterocycle” or “bicyclic heterocyclyl,” as used herein refers to a ring system in which at least one of the ring members is other than carbon. Bicyclic heterocyclyl encompasses ring systems wherein an aromatic ring is fused with another aromatic ring, or wherein an aromatic ring is fused with a non-aromatic ring. Bicyclic heterocyclyl encompasses ring systems wherein a benzene ring is fused to a 5- or a 6-membered ring containing 1, 2 or 3 ring heteroatoms or wherein a pyridine ring is fused to a 5- or a 6-membered ring containing 1, 2 or 3 ring heteroatoms. Bicyclic heterocyclic groups include, but are not limited to, indolyl, indazolyl, pyrazolo[1,5-a]pyridinyl, benzofuranyl, quinolinyl, quinoxalinyl, 1,3-benzodioxolyl, 2,3-dihydro-1,4-benzodioxinyl, 3,4-dihydro-2H-chromenyl, 1H-pyrazolo[4,3-c]pyridin-3-yl; 1H-pyrrolo[3,2-b]pyridin-3-yl; and 1H-pyrazolo[3,2-b]pyridin-3-yl.

The term “heterocycloalkyl” as used herein refers to an aliphatic, partially unsaturated or fully saturated, 3- to 14-membered ring system, including single rings of 3 to 8 atoms and bi- and tricyclic ring systems. The heterocycloalkyl ring-systems include one to four heteroatoms independently selected from oxygen, nitrogen, and sulfur, wherein a nitrogen and sulfur heteroatom optionally can be oxidized and a nitrogen heteroatom optionally can be substituted. Representative heterocycloalkyl groups include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “hydroxyl” or “hydroxyl” as used herein is represented by the formula —OH.

The term “ketone” as used herein is represented by the formula A¹C(O)A², where A¹ and A² can be, independently, an alkyl, cycloalkyl, alkenyl, cycloalkenyl, alkynyl, cycloalkynyl, aryl, or heteroaryl group as described herein.

The term “azide” or “azido” as used herein is represented by the formula —N₃.

The term “nitro” as used herein is represented by the formula —NO₂.

The term “nitrile” or “cyano” as used herein is represented by the formula —CN.

The term “silyl” as used herein is represented by the formula -SiA¹A²A3, where A¹, A², and A³ can be, independently, hydrogen or an alkyl, cycloalkyl, alkoxy, alkenyl, cycloalkenyl, alkynyl, cycloalkynyl, aryl, or heteroaryl group as described herein.

The term “sulfo-oxo” as used herein is represented by the formulas —S(O)A¹, —S(O)₂A¹, —OS(O)₂A¹, or —OS(O)₂OA¹, where A¹ can be hydrogen or an alkyl, cycloalkyl, alkenyl, cycloalkenyl, alkynyl, cycloalkynyl, aryl, or heteroaryl group as described herein. Throughout this specification “S(O)” is a short hand notation for S═O. The term “sulfonyl” is used herein to refer to the sulfo-oxo group represented by the formula —S(O)₂A¹, where A¹ can be hydrogen or an alkyl, cycloalkyl, alkenyl, cycloalkenyl, alkynyl, cycloalkynyl, aryl, or heteroaryl group as described herein. The term “sulfone” as used herein is represented by the formula A¹S(O)₂A², where A¹ and A² can be, independently, an alkyl, cycloalkyl, alkenyl, cycloalkenyl, alkynyl, cycloalkynyl, aryl, or heteroaryl group as described herein. The term “sulfoxide” as used herein is represented by the formula A¹S(O)A², where A and A² can be, independently, an alkyl, cycloalkyl, alkenyl, cycloalkenyl, alkynyl, cycloalkynyl, aryl, or heteroaryl group as described herein.

The term “thiol” as used herein is represented by the formula —SH.

“R¹,” “R²,” “R³,” “Rⁿ,” where n is an integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As described herein, compounds of the invention may contain “optionally substituted” moieties. In general, the term “substituted,” whether preceded by the term “optionally” or not, means that one or more hydrogen of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. In is also contemplated that, in certain aspects, unless expressly indicated to the contrary, individual substituents can be further optionally substituted (i.e., further substituted or unsubstituted).

The term “stable,” as used herein, refers to compounds that are not substantially altered when subjected to conditions to allow for their production, detection, and, in certain aspects, their recovery, purification, and use for one or more of the purposes disclosed herein.

Suitable monovalent substituents on a substitutable carbon atom of an “optionally substituted” group are independently halogen; —(CH₂)₀₋₄R^{◯}; —(CH₂)₀₋₄OR^{◯}; —O(CH₂)₀₋₄R^{◯}, —O—(CH₂)₀₋₄C(O)OR^{◯}; —(CH₂)₀₋₄CH(OR^{◯})₂; —(CH₂)₀₋₄SR^{◯}; —(CH₂)₀₋₄Ph, which may be substituted with R^{◯}; —(CH₂)₀₋₄O(CH₂)₀₋₁Ph which may be substituted with R^{◯}; —CH═CHPh, which may be substituted with R^{◯}; —(CH₂)₀₋₄O(CH₂)₀₋₁-pyridyl which may be substituted with R^{◯}; —NO₂; —CN; —N₃; —(CH₂)₀₋₄N(R^{◯})₂; —(CH₂)₀₋₄N(R^{◯})C(O)R^{◯}; —N(R^{◯})C(S)R^{◯}; —(CH₂)₀₋₄N(R^{◯})C(O)NR^{◯}₂; —N(R^{◯})C(S)NR^{◯}₂; —(CH₂)₀₋₄N(R^{◯})C(O)OR^{◯}; —N(R^{◯})N(R^{◯})C(O)R^{◯}; —N(R^{◯})N(R^{◯})C(O)NR^{◯}₂; —N(R^{◯})N(R^{◯})C(O)OR^{◯}; —(CH₂)₀₋₄C(O)R^{◯}; —C(S)R^{◯}; —(CH₂)₀₋₄C(O)OR^{◯}; —(CH₂)₀₋₄C(O)SR^{◯}; —(CH₂)₀₋₄C(O)OSiR^{◯}₃; —(CH₂)₀₋₄OC(O)R^{◯}; —OC(O)(CH₂)₀₋₄SR—, SC(S)SR^{◯}; —(CH₂)₀₋₄SC(O)R^{◯}; —(CH₂)₀₋₄C(O)NR^{◯}₂; —C(S)NR^{◯}₂; —C(S)SR^{◯}; —(CH₂)₀₋₄OC(O)NR^{◯}₂; —C(O)N(OR^{◯})R^{◯}; —C(O)C(O)R^{◯}; —C(O)CH₂C(O)R^{◯}; —C(NOR^{◯})R^{◯}; —(CH₂)₀₋₄SSR^{◯}; —(CH₂)₀₋₄S(O)₂R^{◯}; —(CH₂)₀₋₄S(O)₂OR^{◯}; —(CH₂)₀₋₄OS(O)₂R^{◯}; —S(O)₂NR^{◯}₂; —(CH₂)₀₋₄S(O)R^{◯}; —N(R^{◯})S(O)₂NR^{◯}₂; —N(R^{◯})S(O)₂R^{◯}; —N(OR^{◯})R^{◯}; —C(NH)NR^{◯}₂; —P(O)₂R^{◯}; —P(O)R^{◯}₂; —OP(O)R^{◯}₂; —OP(O)(OR^{◯})₂; SiR^{◯}₃; —(C₁₋₄ straight or branched alkylene)O—N(R^{◯})₂; or —(C₁₋₄ straight or branched alkylene)C(O)O—N(R^{◯})₂, wherein each R^{◯} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{◯}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{◯} (or the ring formed by taking two independent occurrences of R^{◯} together with their intervening atoms), are independently halogen, —(CH₂)₀₋₂R^{●}, -(haloR^{●}), —(CH₂)₀₋₂H, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{◯} include═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R^{●} include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{◯}, -(haloR*), —OH, —OR*, —O(haloR^{◯}), —CN, —C(O)OH, —C(O)OR*, —NH₂, —NHR^{◯}, —NR^{◯}₂, or —NO₂, wherein each R^{◯} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “leaving group” refers to an atom (or a group of atoms) with electron withdrawing ability that can be displaced as a stable species, taking with it the bonding electrons. Examples of suitable leaving groups include halides and sulfonate esters, including, but not limited to, triflate, mesylate, tosylate, and brosylate.

The terms “hydrolysable group” and “hydrolysable moiety” refer to a functional group capable of undergoing hydrolysis, e.g., under basic or acidic conditions. Examples of hydrolysable residues include, without limitation, acid halides, activated carboxylic acids, and various protecting groups known in the art (see, for example, “Protective Groups in Organic Synthesis,” T. W. Greene, P. G. M. Wuts, Wiley-Interscience, 1999).

The term “organic residue” defines a carbon-containing residue, i.e., a residue comprising at least one carbon atom, and includes but is not limited to the carbon-containing groups, residues, or radicals defined hereinabove. Organic residues can contain various heteroatoms, or be bonded to another molecule through a heteroatom, including oxygen, nitrogen, sulfur, phosphorus, or the like. Examples of organic residues include but are not limited alkyl or substituted alkyls, alkoxy or substituted alkoxy, mono or di-substituted amino, amide groups, etc. Organic residues can preferably comprise 1 to 18 carbon atoms, 1 to 15, carbon atoms, 1 to 12 carbon atoms, 1 to 8 carbon atoms, 1 to 6 carbon atoms, or 1 to 4 carbon atoms. In a further aspect, an organic residue can comprise 2 to 18 carbon atoms, 2 to 15, carbon atoms, 2 to 12 carbon atoms, 2 to 8 carbon atoms, 2 to 4 carbon atoms, or 2 to 4 carbon atoms.

A very close synonym of the term “residue” is the term “radical,” which as used in the specification and concluding claims, refers to a fragment, group, or substructure of a molecule described herein, regardless of how the molecule is prepared. For example, a 2,4-thiazolidinedione radical in a particular compound has the structure:

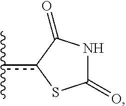

regardless of whether thiazolidinedione is used to prepare the compound. In some embodiments the radical (for example an alkyl) can be further modified (i.e., substituted alkyl) by having bonded thereto one or more “substituent radicals.” The number of atoms in a given radical is not critical to the present invention unless it is indicated to the contrary elsewhere herein.

“Organic radicals,” as the term is defined and used herein, contain one or more carbon atoms. An organic radical can have, for example, 1-26 carbon atoms, 1-18 carbon atoms, 1-12 carbon atoms, 1-8 carbon atoms, 1-6 carbon atoms, or 1-4 carbon atoms. In a further aspect, an organic radical can have 2-26 carbon atoms, 2-18 carbon atoms, 2-12 carbon atoms, 2-8 carbon atoms, 2-6 carbon atoms, or 2-4 carbon atoms. Organic radicals often have hydrogen bound to at least some of the carbon atoms of the organic radical. One example, of an organic radical that comprises no inorganic atoms is a 5, 6, 7, 8-tetrahydro-2-naphthyl radical. In some embodiments, an organic radical can contain 1-10 inorganic heteroatoms bound thereto or therein, including halogens, oxygen, sulfur, nitrogen, phosphorus, and the like. Examples of organic radicals include but are not limited to an alkyl, substituted alkyl, cycloalkyl, substituted cycloalkyl, mono-substituted amino, di-substituted amino, acyloxy, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy, haloalkyl, haloalkoxy, aryl, substituted aryl, heteroaryl, heterocyclic, or substituted heterocyclic radicals, wherein the terms are defined elsewhere herein. A few non-limiting examples of organic radicals that include heteroatoms include alkoxy radicals, trifluoromethoxy radicals, acetoxy radicals, dimethylamino radicals and the like.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and l or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Ingold-Prelog system can be used to assign the (R) or (S) configuration to a chiral carbon.

When the disclosed compounds contain one chiral center, the compounds exist in two enantiomeric forms. Unless specifically stated to the contrary, a disclosed compound includes both enantiomers and mixtures of enantiomers, such as the specific 50:50 mixture referred to as a racemic mixture. The enantiomers can be resolved by methods known to those skilled in the art, such as formation of diastereoisomeric salts which may be separated, for example, by crystallization (see, CRC Handbook of Optical Resolutions via Diastereomeric Salt Formation by David Kozma (CRC Press, 2001)); formation of diastereoisomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. It will be appreciated that where the desired enantiomer is converted into another chemical entity by one of the separation procedures described above, a further step can liberate the desired enantiomeric form. Alternatively, specific enantiomers can be synthesized by asymmetric synthesis using optically active reagents, substrates, catalysts or solvents, or by converting one enantiomer into the other by asymmetric transformation.

Designation of a specific absolute configuration at a chiral carbon in a disclosed compound is understood to mean that the designated enantiomeric form of the compounds can be provided in enantiomeric excess (e.e.). Enantiomeric excess, as used herein, is the presence of a particular enantiomer at greater than 50%, for example, greater than 60%, greater than 70%, greater than 75%, greater than 80%, greater than 85%, greater than 90%, greater than 95%, greater than 98%, or greater than 99%. In one aspect, the designated enantiomer is substantially free from the other enantiomer. For example, the “R” forms of the compounds can be substantially free from the “S” forms of the compounds and are, thus, in enantiomeric excess of the “S” forms. Conversely, “S” forms of the compounds can be substantially free of “R” forms of the compounds and are, thus, in enantiomeric excess of the “R” forms.

When a disclosed compound has two or more chiral carbons, it can have more than two optical isomers and can exist in diastereoisomeric forms. For example, when there are two chiral carbons, the compound can have up to four optical isomers and two pairs of enantiomers ((S,S)/(R,R) and (R,S)/(S,R)). The pairs of enantiomers (e.g., (S,S)/(R,R)) are mirror image stereoisomers of one another. The stereoisomers that are not mirror-images (e.g., (S,S) and (R,S)) are diastereomers. The diastereoisomeric pairs can be separated by methods known to those skilled in the art, for example chromatography or crystallization and the individual enantiomers within each pair may be separated as described above. Unless otherwise specifically excluded, a disclosed compound includes each diastereoisomer of such compounds and mixtures thereof.

The compounds according to this disclosure may form prodrugs at hydroxyl or amino functionalities using alkoxy, amino acids, etc., groups as the prodrug forming moieties. For instance, the hydroxymethyl position may form mono-, di- or triphosphates and again these phosphates can form prodrugs. Preparations of such prodrug derivatives are discussed in various literature sources (examples are: Alexander et al., J. Med. Chem. 1988, 31, 318; Aligas-Martin et al., PCT WO 2000/041531, p. 30). The nitrogen function converted in preparing these derivatives is one (or more) of the nitrogen atoms of a compound of the disclosure.

“Derivatives” of the compounds disclosed herein are pharmaceutically acceptable salts, prodrugs, deuterated forms, radioactively labeled forms, isomers, solvates and combinations thereof. The “combinations” mentioned in this context are refer to derivatives falling within at least two of the groups: pharmaceutically acceptable salts, prodrugs, deuterated forms, radioactively labeled forms, isomers, and solvates. Examples of radioactively labeled forms include compounds labeled with tritium, phosphorous-32, iodine-129, carbon-11, fluorine-18, among others, as disclosed elsewhere herein.

Compounds described herein comprise atoms in both their natural isotopic abundance and in non-natural abundance. The disclosed compounds can be isotopically labeled or isotopically substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, phosphorous, fluorine and chlorine, such as²H, ³H, ¹³C, ¹⁴C, ¹⁵N, ¹⁸O, ¹⁷O, ³⁵S, ¹⁸F and ³⁶Cl, respectively. Compounds further comprise prodrugs thereof, and pharmaceutically acceptable salts of said compounds or of said prodrugs which contain the aforementioned isotopes and/or other isotopes of other atoms are within the scope of this invention. Certain isotopically labeled compounds of the present invention, for example those into which radioactive isotopes such as PET, SPECT, therapeutic radionuclides, ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of the present invention and prodrugs thereof can generally be prepared by carrying out the procedures below, by substituting a readily available isotopically labeled reagent for a non-isotopically labeled reagent.

The compounds described in the invention can be present as a solvate. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvent or water molecules can combine with the compounds according to the invention to form solvates and hydrates.

Unless stated to the contrary, the invention includes all such possible solvates.

The term “co-crystal” means a physical association of two or more molecules that owe their stability through non-covalent interaction. One or more components of this molecular complex provide a stable framework in the crystalline lattice. In certain instances, the guest molecules are incorporated in the crystalline lattice as anhydrates or solvates, see e.g. “Crystal Engineering of the Composition of Pharmaceutical Phases. Do Pharmaceutical Co-crystals Represent a New Path to Improved Medicines?” Almarasson, O., et. al., The Royal Society of Chemistry, 1889-1896, 2004. Examples of co-crystals include p-toluenesulfonic acid and benzenesulfonic acid.

It is also appreciated that certain compounds described herein can be present as an equilibrium of tautomers. For example, ketones with an α-hydrogen can exist in an equilibrium of the keto form and the enol form.

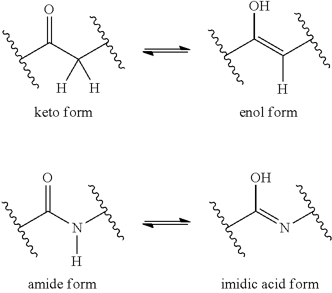 [SMILES: *CC(*)=O ; */C=C(/*)O ; *NC(*)=O ; */N=C(/*)O]

Likewise, amides with an N-hydrogen can exist in an equilibrium of the amide form and the imidic acid form. As another example, pyrazoles can exist in two tautomeric forms, N¹-unsubstituted, 3-A³ and N¹-unsubstituted, 5-A³ as shown below.

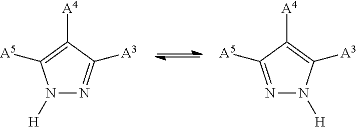

Unless stated to the contrary, the invention includes all such possible tautomers.

It is known that chemical substances form solids that are present in different states of order that are termed polymorphic forms or modifications. The different modifications of a polymorphic substance can differ greatly in their physical properties. The compounds according to the invention can be present in different polymorphic forms, with it being possible for particular modifications to be metastable. Unless stated to the contrary, the invention includes all such possible polymorphic forms.

In some aspects, a structure of a compound can be represented by a formula:

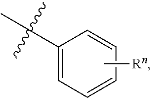

which is understood to be equivalent to a formula:

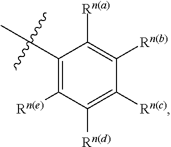

wherein n is typically an integer. That is, Rⁿ is understood to represent five independent substituents, R^{n(a)}, R^{n(b)}, R^{n(c)}, R^{n(d)}, R^{n(e)}. By “independent substituents,” it is meant that each R substituent can be independently defined. For example, if in one instance R^{n(a)} is halogen, then R^{n(b)} is not necessarily halogen in that instance.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Strem Chemicals (Newburyport, Mass.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and supplemental volumes (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

### B. Compounds

In one aspect, the invention relates to compounds useful in treating disorders associated with modification of SGLT expression such as, for example, disorders of uncontrolled cellular proliferation, neurological disorders, atherosclerosiss, diabetes, and heart disease.

In one aspect, the compounds of the invention are useful in inhibiting SGLT expression in a mammal. In a further aspect, the compounds of the invention are useful in inhibiting SGLT expression in at least one cell.

In one aspect, the compounds of the invention are useful in knocking-down or knocking-up SGLT expression in a mammal. In a further aspect, the compounds of the invention are useful in knocking-down or knocking-up SGLT expression in at least one cell.

In one aspect, the compounds of the invention are useful in the treatment of disorders of uncontrolled cellular proliferation, as further described herein.

In one aspect, the compounds of the invention are useful in the treatment of neurological disorders, as further described herein.

In one aspect, the compounds of the invention are useful in the treatment of atherosclerosis and/or heart disease, as further described herein.

In one aspect, the compounds of the invention are useful in the treatment of diabetes, as further described herein.

It is contemplated that each disclosed derivative can be optionally further substituted. It is also contemplated that any one or more derivative can be optionally omitted from the invention. It is understood that a disclosed compound can be provided by the disclosed methods. It is also understood that the disclosed compounds can be employed in the disclosed methods of using.

1. Structure

In one aspect, disclosed are compounds having a structure represented by a formula:

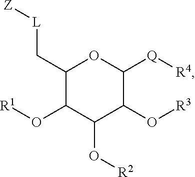

wherein L is a divalent linker; wherein Q is selected from —O—, —NHC(O)—, and —NHC(S)NH—; wherein Z is a fluorophore, a radiolabel, a radioisotope, or a radiotherapeutic; and wherein each of R¹, R², R³, and R⁴ is independently selected from hydrogen, C1-C4 alkyl, aryl, a fluorophore residue, a peptide residue, and a polyaromatic residue, provided that when Q is —O— and Z is a fluorophore, then at least one of R¹, R², R³, and R⁴ is not hydrogen, or a pharmaceutically acceptable salt thereof.

In a further aspect, disclosed are compounds having a structure represented by a formula:

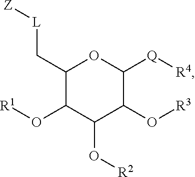

wherein L is a divalent linker; wherein Q is selected from —O—, —NHC(O)—, and —NHC(S)NH—; wherein Z is a therapeutic compound, such as a chemotherapeutic; and wherein each of R¹, R², R³, and R⁴ is independently selected from hydrogen, C1-C4 alkyl, aryl, a fluorophore residue, a peptide residue, and a polyaromatic residue, or a pharmaceutically acceptable salt thereof. Examples of suitable therapeutic compounds that can be encompassed within Z include without limitation anti-cancer antibiotics, such as doxorubicin among others. A non-limiting example is a compound having the following structure, which includes a doxorubicin conjugate as Z:

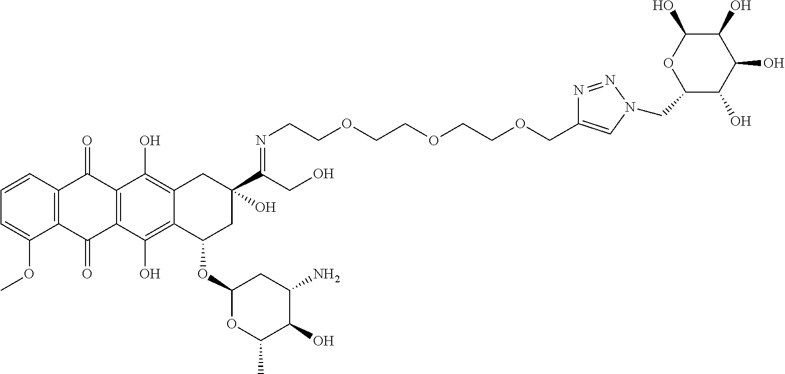 [SMILES: COc1cccc2c1C(=O)c1c(O)c3c(c(O)c1C2=O)C[C@@](O)(/C(CO)=N/CCOCCOCCOCc1cn(C[C@@H]2O[C@@H](O)[C@@H](O)[C@@H](O)[C@@H]2O)nn1)C[C@@H]3O[C@H]1C[C@H](N)[C@@H](O)[C@H](C)O1]

In various aspects, Q is —O—, Z is a fluorophore, and at least two of R¹, R², R³, and R⁴ are not hydrogen.

In various aspects, Z is a fluorophore having a structure represented by a formula:

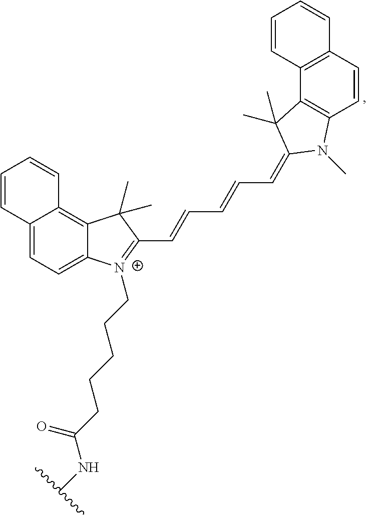 [SMILES: *NC(=O)CCCCC[N+]1=C(/C=C/C=C/C=C2/N(C)c3ccc4ccccc4c3C2(C)C)C(C)(C)c2c1ccc1ccccc21]

and R⁴ is not hydrogen.

In a further aspect, the compound has a structure represented by a formula:

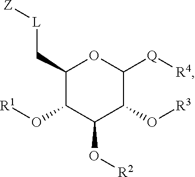

In a further aspect, the compound has a structure represented by a formula:

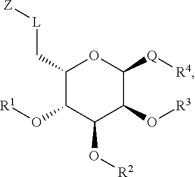

In a further aspect, the compound has a structure represented by a formula:

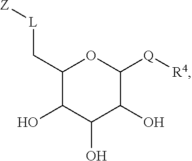

In a further aspect, the compound has a structure represented by a formula:

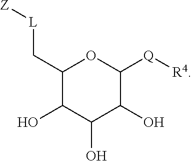

In a further aspect, the compound has a structure represented by a formula:

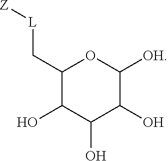

In a further aspect, the compound has a structure represented by a formula:

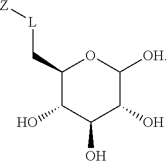

In a further aspect, the compound has a structure represented by a formula:

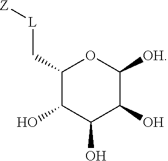

In a further aspect, the compound has a structure represented by a formula:

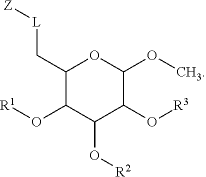

In a further aspect, the compound has a structure represented by a formula:

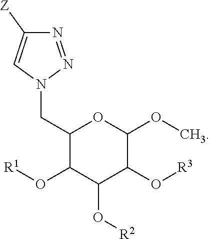

In a further aspect, the compound has a structure represented by a formula:

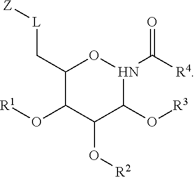

In a further aspect, the compound has a structure represented by a formula:

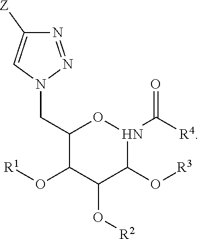

In a further aspect, the compound has a structure represented by a formula:

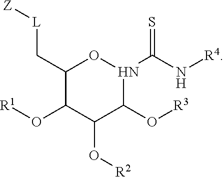

In a further aspect, the compound has a structure represented by a formula:

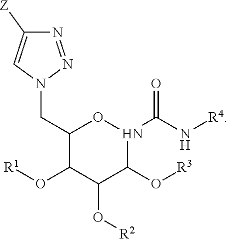

In a further aspect, the compound has a structure represented by a formula selected from:

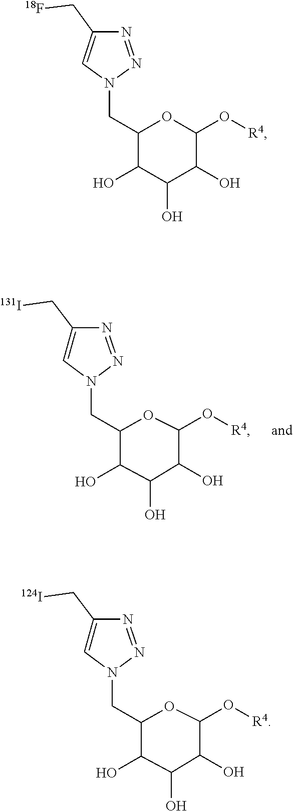

In a further aspect, the compound has a structure represented by a formula selectedfrom:

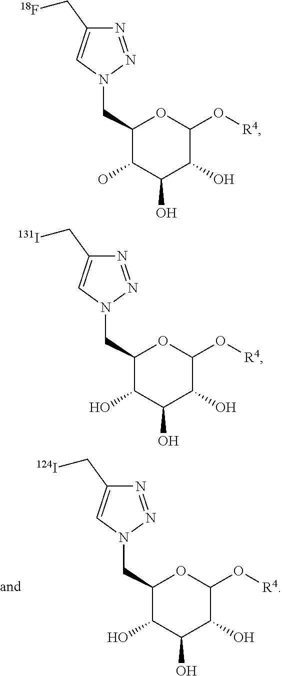

In a further aspect, the compound has a structure selected from:

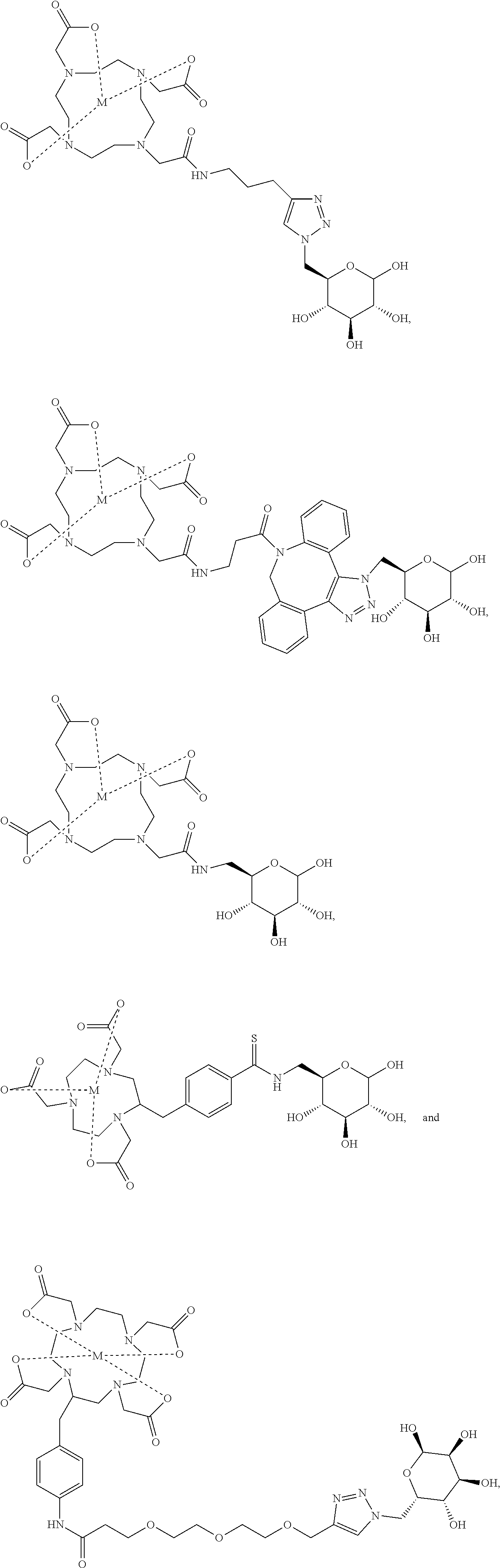

wherein M is a radioisotope. Examples of radioisotopes include, but are not limited to ⁶⁸Ga, ⁶⁴Cu, ¹⁷⁷Lu, ⁸⁹Zr, ⁸⁶Y (beta+), ⁹⁰Y (beta−), ⁴³Sc, ⁴⁴Sc, ¹⁵²Tb, ⁸²Rb, ²²⁵Ac, ²¹¹At, ²²⁷Th (CTEP), ²¹³Bi, ²²⁴Ra, ²²³Ra (CTEP), ⁸⁹Sr, and ¹⁵³Sm.

a. L Groups

In one aspect, L is a divalent linker. Examples of divalent linkers include, but are not limited to:

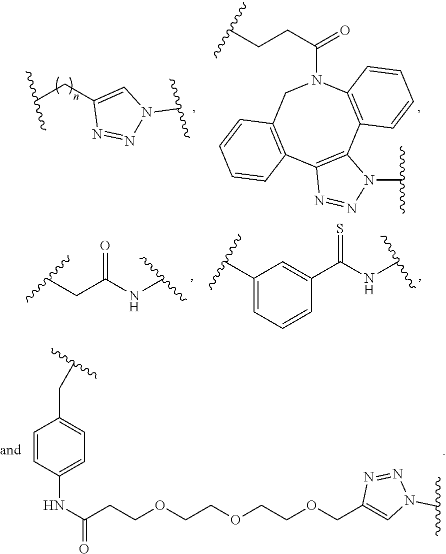

In a further aspect, L comprises a triazole, an amide, a thioamide, a peptide, and polyethylene glycol (PEG) residue.

In a further aspect, L comprises a triazole. In a still further aspect, L has a structure represented by a formula selected from:

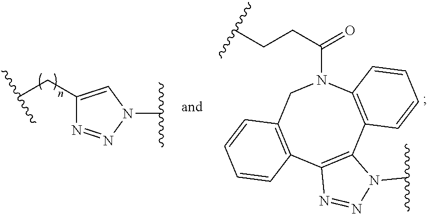

wherein n is 1, 2, 3, or 4. In yet a further aspect, n is 1, 2, or 3. In an even further aspect, n is 1 or 2. In a still further aspect, n is 4. In yet a further aspect, n is 3. In an even further aspect, n is 2. In a still further aspect, n is 1.

In a further aspect, L comprises an amide. In a still further aspect, L has a structure represented by a formula:

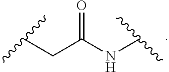 [SMILES: *CC(=O)N*]

In a further aspect, L comprises a thioamide. In a still further aspect, L has a structure represented by a formula:

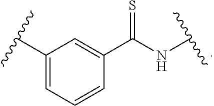 [SMILES: *NC(=S)c1cccc(*)c1]

In a further aspect, L comprises a peptide. Examples of peptides include, but are not limited to, HRH agonists and synthetic analogs, leuprolide, somatostatin analogs, hormones, octreotides, glucagons-like peptides, oxytocins, and the like.

In a further aspect, L comprises a PEG residue.

b. Q Groups

In one aspect, Q is selected from —O—, —NHC(O)—, and —NHC(S)NH—. In a further aspect, Q is selected from —NHC(O)— and —NHC(S)NH—. In a still further aspect, Q is —NHC(O)—. In yet a further aspect, Q is —NHC(S)NH—.

In a further aspect, Q is —O—.

c. Z Groups

In one aspect, Z is a fluorophore, a radiolabel, a radioisotope, or a radiotherapeutic. In a further aspect, Z is a radiolabel, a radioisotope, or a radiotherapeutic.

In a further aspect, Z is a radiolabel. In a still further aspect, the radiolabel comprises a ligand and a metal. In yet a further aspect, the ligand is selected from DOTA, NOTA, DFO (9H-cyclopenta[1,2-b:4,3-b′]dipyridin-9-one), and DOTA-DBCO. In an even further aspect, the metal is selected from ⁶⁸Ga, ⁶⁴Cu, ¹⁷⁷Lu, ⁸⁹Zr, ⁸⁶Y ⁹⁰Y, ⁴³Sc, ⁴⁴Sc, ¹⁵²Tb, ⁸²Rb, ²²⁵Ac, ²¹¹At, ²²⁷Th, ²¹³Bi, ²²⁴Ra, ²²³Ra, ⁸⁹Sr, ¹⁵³Sm, and the like.

In a further aspect, Z is a radioisotope. Examples of radioisotopes include, but are not limited to ¹⁸F, ¹²⁴I, and ¹³¹I.

In a further aspect, Z is a radiotherapeutic.

In a further aspect, Z is a fluorophore. In a still further aspect, the fluorophore is a residue of a rhodamine or a cyanine. In yet a further aspect, the fluorophore has a structure represented by a formula selected from:

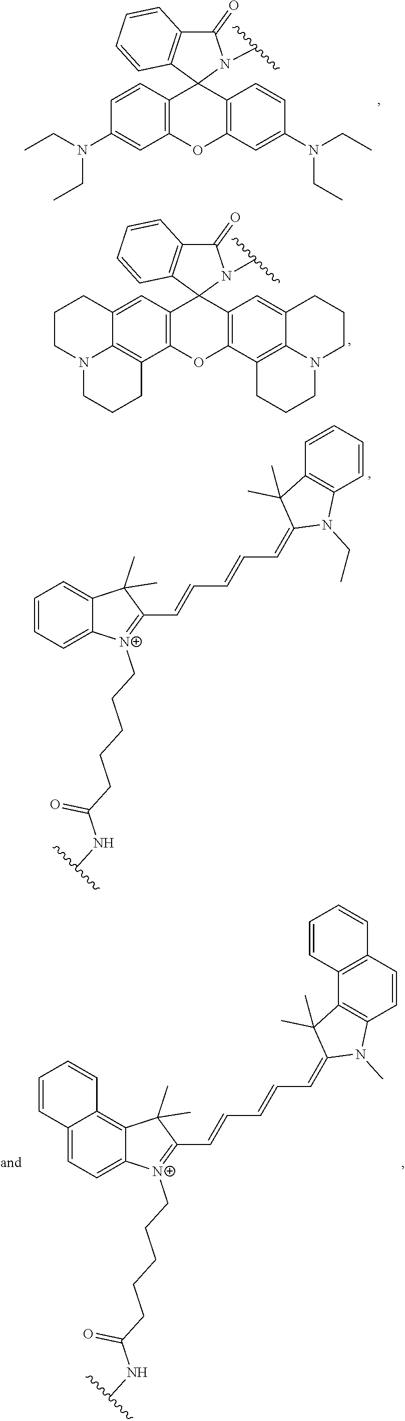 [SMILES: *N1C(=O)c2ccccc2C12c1ccc(N(CC)CC)cc1Oc1cc(N(CC)CC)ccc12 ; *N1C(=O)c2ccccc2C12c1cc3c4c(c1Oc1c2cc2c5c1CCCN5CCC2)CCCN4CCC3 ; *NC(=O)CCCCC[N+]1=C(/C=C/C=C/C=C2/N(CC)c3ccccc3C2(C)C)C(C)(C)c2ccccc21 ; *NC(=O)CCCCC[N+]1=C(/C=C/C=C/C=C2/N(C)c3ccc4ccccc4c3C2(C)C)C(C)(C)c2c1ccc1ccccc21]

In an even further aspect, the fluorophore has a structure represented by a formula:

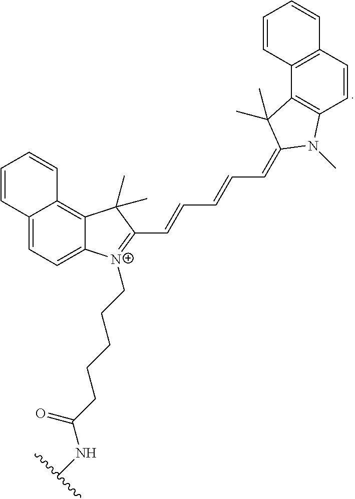 [SMILES: *NC(=O)CCCCC[N+]1=C(/C=C/C=C/C=C2/N(C)c3ccc4ccccc4c3C2(C)C)C(C)(C)c2c1ccc1ccccc21]

d. R¹, R², R³, and R⁴ Groups

In one aspect, each of R¹, R², R³, and R⁴ is independently selected from hydrogen, C1-C4 alkyl, aryl, a fluorophore residue, a peptide residue, and a polyaromatic residue, provided that when Q is —O— and Z is a fluorophore, then at least one of R¹, R², R³, and R⁴ is not hydrogen. In a further aspect, each of R¹, R², R³, and R⁴ is hydrogen.

In a further aspect, each of R¹, R², R³, and R⁴ is independently selected from hydrogen and C1-C4 alkyl. In a still further aspect, each of R¹, R², R³, and R⁴ is independently selected from hydrogen, methyl, ethyl, n-propyl, and isopropyl. In yet a further aspect, each of R¹, R², R³, and R⁴ is independently selected from hydrogen, methyl, and ethyl. In an even further aspect, each of R¹, R², R³, and R⁴ is independently selected from hydrogen and ethyl. In a still further aspect, each of R¹, R², R³, and R⁴ is independently selected from hydrogen and methyl.

In a further aspect, each of R¹, R², R³, and R⁴ is independently selected from hydrogen and aryl. Examples of aryls include, but are not limited to, phenyl, naphthyl, phenanthryl, and anthracenyl. In a still further aspect, each of R¹, R², R³, and R⁴ is independently selected from hydrogen and aryl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, each of R¹, R², R³, and R⁴ is independently selected from hydrogen and aryl substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, each of R¹, R², R³, and R⁴ is independently selected from hydrogen and aryl substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, each of R¹, R², R³, and R⁴ is independently selected from hydrogen and aryl monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, each of R¹, R², R³, and R⁴ is independently selected from hydrogen and unsubstituted aryl.

In a further aspect, each of R¹, R², R³, and R⁴ is independently selected from hydrogen and phenyl. In a still further aspect, each of R¹, R², R³, and R⁴ is independently selected from hydrogen and phenyl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, each of R¹, R², R³, and R⁴ is independently selected from hydrogen and phenyl substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, each of R, R², R³, and R⁴ is independently selected from hydrogen and phenyl substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, each of R, R², R³, and R⁴ is independently selected from hydrogen and phenyl monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, each of R¹, R², R³, and R⁴ is independently selected from hydrogen and unsubstituted phenyl.

In a further aspect, each of R, R², R³, and R⁴ is independently selected from hydrogen and heteroaryl. Examples of heteroaryls include, but are not limited to, pyrrole, furan, thiophene, pyridine, pyridazine, pyrimidine, pyrazine, triazine, indole, indazole, benzimidazole, azaindazole, purine, benzofuran, benzo[b]thiophene, benzo[d]oxazole, and benzo[d]isothiazole. In a still further aspect, each of R, R², R³, and R⁴ is independently selected from hydrogen and heteroaryl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, each of R, R², R³, and R⁴ is independently selected from hydrogen and heteroaryl substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, each of R, R², R³, and R⁴ is independently selected from hydrogen and heteroaryl substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, each of R¹, R², R³, and R⁴ is independently selected from hydrogen and heteroaryl monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, each of R¹, R², R³, and R⁴ is independently selected from hydrogen and unsubstituted heteroaryl.

In a further aspect, each of R¹, R², R³, and R⁴ is independently selected from hydrogen and a fluorophore residue. Examples of fluorophores include, but are not limited to, 2,4-Bis[1,3,3-trimethyl-2-indolinylidenemethyl]cyclobutenediylium-1,3-dioxolate, 2,4 Bis[3,3-dimethyl-2-(1H-benz[e]indolinylidenemethyl)]cyclobutenediylium-1,3-dioxolate, 2,4-Bis[3,5-dimethyl-2-pyrrolyl]cyclobutenediylium-1,3-diololate, quantum dots, Alexa Fluor® dyes, AMCA, BODIPY® 630/650, BODIPY® 650/665, BODIPY®-FL, BODIPY®-R⁶G, BODIPY®-TMR, BODIPY®-TRX, Cascade Blue®, CyDye™, including but not limited to Cy2™, Cy3™, and Cy5™, a DNA intercalating dye, 6-FAM™, Fluorescein, HEX™, 6-JOE, Oregon Green® 488, Oregon Green® 500, Oregon Green® 514, Pacific Blue™, REG, phycobilliproteins including, but not limited to, phycoerythrin and allophycocyanin, Rhodamine Green™, Rhodamine Red™, ROX™, TAMRA™, TET™, Tetramethylrhodamine, and Texas Red®.

In a further aspect, each of R¹, R², R³, and R⁴ is independently selected from hydrogen and a peptide residue. Examples of peptide residues include, but are not limited to, residues of HRH agonists and synthetic analogs, leuprolide, somatostatin analogs, hormones, octreotides, glucagons-like peptides, oxytocins, and the like.

In a further aspect, each of R¹, R², R³, and R⁴ is independently selected from hydrogen and a polyaromatic residue. Examples of polyaromatic residues include polymers formed from one or more aromatic monomers such as, for example, vinyl unsubstituted aromatic monomers (e.g., styrene, 2-vinyl naphthalene), vinyl substituted aromatics (e.g., alpha-methyl styrene), ring-substituted vinyl aromatics (e.g., 3-methylstyrene, 4-methylstyrene, 2,4-dimethylstyrene, 2,5-dimethylstyrene, 3,5-dimethylstyrene, 2,4,6-trimethylstyrene, and 4-tert-butylstyrene), ring-alkoxylated vinyl aromatics (e.g., 4-methoxystyrene, 4-ethoxystyrene), ring-halogenated vinyl aromatics (e.g., 2-chlorostyrene, 3-chlorostyrene, 4-chlorostyrene, 2,6-dichlorostyrene, 4-bromostyrene, and 4-fluorostyrene), ring-ester-substituted vinyl aromatics (e.g., 4-acetoxystyrene), ring-hydroxylated vinyl aromatics (e.g., 4-hydroxystyrene), ring-amino-substituted vinyl aromatics (e.g., 4-amino styrene), ring-silyl-substituted styrenes (e.g., p-dimethylethoxy siloxy styrene), unsubstituted and substituted vinyl pyridines (e.g., 2-vinyl pyridine, 4-vinyl pyridine), vinyl aromatic esters (e.g., vinyl benzoate, vinyl 4-tert-butyl benzoate), other vinyl aromatic monomers (e.g., vinyl carbazole, vinyl ferrocene), aromatic acrylates (e.g., benzyl acrylate), and aromatic methacrylates (e.g., phenyl methacrylate, benzyl methacrylate).

e. Example Compounds

In one aspect, a compound is selected from:

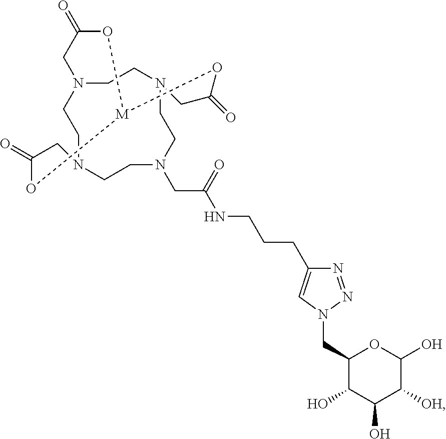

wherein M is a radioisotope such as, for example, ⁶⁸Ga, ⁶⁴Cu, ¹⁷⁷Lu, ⁸⁹Zr, ⁸⁶Y ⁹⁰Y, ⁴³Sc, ⁴⁴Sc, ¹⁵²Tb, ⁸²Rb, ²²⁵Ac, ²¹¹At, ²²⁷Th, ²¹³Bi, ²²⁴Ra, ²²³Ra, ⁸⁹Sr, and ¹⁵³Sm, or a pharmaceutically acceptable salt thereof.

In one aspect, a compound is selected from:

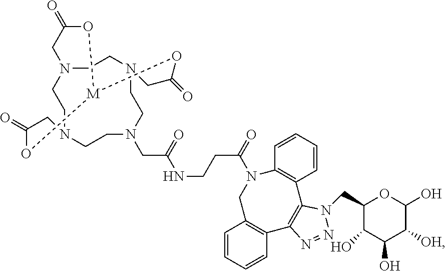

wherein M is a radioisotope such as, for example, ⁶⁸Ga, ⁶⁴Cu, ¹⁷⁷Lu, ⁸⁹Zr, ⁸⁶Y, ⁹⁰Y, ⁴³Sc, ⁴⁴Sc, ¹⁵²Tb, ⁸²Rb, ²²⁵Ac, ²¹¹At, ²²⁷Th, ²¹³Bi, ²²⁴Ra, ²²³Ra, ⁸⁹Sr, and ¹⁵³Sm, or a pharmaceutically acceptable salt thereof.

In one aspect, a compound is selected from:

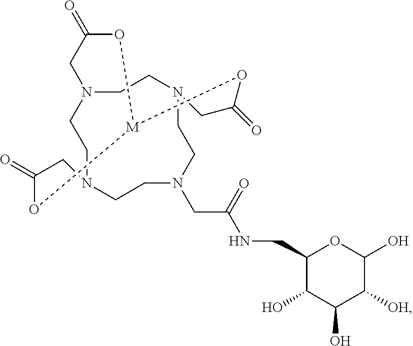

wherein M is a radioisotope such as, for example, ⁶⁸Ga, ⁶⁴Cu, ¹⁷⁷Lu, ⁸⁹Zr, ⁸⁶Y, 9Y, ⁴³Sc, ⁴⁴Sc, ¹⁵²Tb, ⁸²Rb, ²²⁵Ac, ²¹¹At, ²²⁷Th, ²¹³Bi, ²²⁴Ra, ²²³Ra, ⁸⁹Sr, and ¹⁵³Sm, or a pharmaceutically acceptable salt thereof.

In one aspect, a compound is selected from:

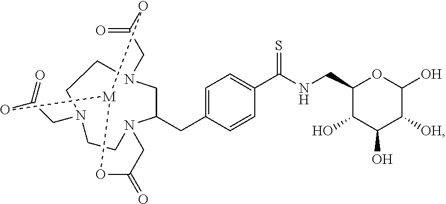

wherein M is a radioisotope such as, for example, ⁶⁸Ga, ⁶⁴Cu, ⁷⁷Lu, ⁸⁹Zr, ⁸⁶Y, ⁹⁰Y ⁴³Sc, ⁴⁴Sc, ¹⁵²Tb, ⁸²Rb, ²²⁵Ac, ²¹¹At, ²²⁷Th, ²¹³Bi, ²²⁴Ra, ²²³Ra, ⁸⁹Sr, and ¹⁵³Sm, or a pharmaceutically acceptable salt thereof.

In one aspect, a compound is selected from:

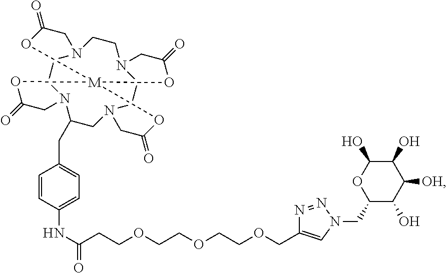

wherein M is a radioisotope such as, for example, ⁶⁸Ga, ⁶⁴Cu, ¹⁷⁷Lu, ⁸⁹Zr, ⁸⁶Y, ⁹⁰Y, ⁴³Sc, ⁴⁴Sc, ¹⁵²Tb, ⁸²Rb, ²²⁵Ac, ²¹¹At, ²²⁷Th, ²¹³Bi, ²²⁴Ra, ²²³Ra, ⁸⁹Sr, and ¹⁵³Sm, or a pharmaceutically acceptable salt thereof.

In one aspect, a compound is selected from:

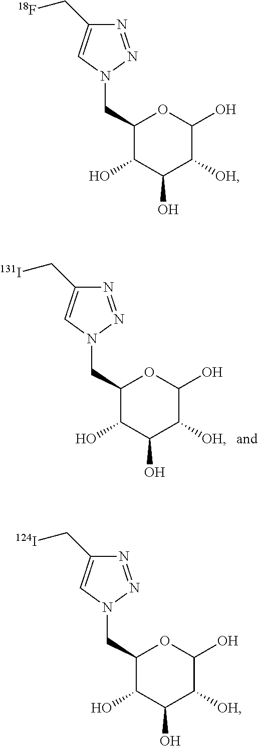 [SMILES: OC1O[C@H](Cn2cc(C[18F])nn2)[C@@H](O)[C@H](O)[C@H]1O ; OC1O[C@H](Cn2cc(C[131I])nn2)[C@@H](O)[C@H](O)[C@H]1O ; OC1O[C@H](Cn2cc(C[124I])nn2)[C@@H](O)[C@H](O)[C@H]1O]

or a pharmaceutically acceptable salt thereof.

### C. Pharmaceutical Compositions

In one aspect, disclosed are pharmaceutical compositions comprising a disclosed compound, or a pharmaceutically acceptable salt thereof, and a pharmaceutically acceptable carrier.

In one aspect, disclosed are pharmaceutical compositions comprising a pharmaceutically acceptable carrier and a therapeutically effective amount of at least one compound having a structure represented by a formula:

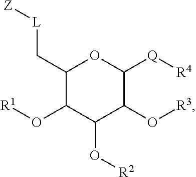

wherein L is a divalent linker; wherein Q is selected from —O—, —NHC(O)—, and —NHC(S)NH—; wherein Z is a fluorophore, a radiolabel, a radioisotope, or a radiotherapeutic; and wherein each of R¹, R², R³, and R⁴ is independently selected from hydrogen, C1-C4 alkyl, aryl, a fluorophore residue, a peptide residue, and a polyaromatic residue, provided that when Q is —O— and Z is a fluorophore, then at least one of R¹, R², R³, and R⁴ is not hydrogen, or a pharmaceutically acceptable salt thereof.

In various aspects, the compounds and compositions of the invention can be administered in pharmaceutical compositions, which are formulated according to the intended method of administration. The compounds and compositions described herein can be formulated in a conventional manner using one or more physiologically acceptable carriers or excipients. For example, a pharmaceutical composition can be formulated for local or systemic administration, e.g., intravenous, topical, or oral administration.

The nature of the pharmaceutical compositions for administration is dependent on the mode of administration and can readily be determined by one of ordinary skill in the art. In various aspects, the pharmaceutical composition is sterile or sterilizable. The therapeutic compositions featured in the invention can contain carriers or excipients, many of which are known to skilled artisans. Excipients that can be used include buffers (for example, citrate buffer, phosphate buffer, acetate buffer, and bicarbonate buffer), amino acids, urea, alcohols, ascorbic acid, phospholipids, polypeptides (for example, serum albumin), EDTA, sodium chloride, liposomes, mannitol, sorbitol, water, and glycerol. The nucleic acids, polypeptides, small molecules, and other modulatory compounds featured in the invention can be administered by any standard route of administration. For example, administration can be parenteral, intravenous, subcutaneous, or oral. A modulatory compound can be formulated in various ways, according to the corresponding route of administration. For example, liquid solutions can be made for administration by drops into the ear, for injection, or for ingestion; gels or powders can be made for ingestion or topical application. Methods for making such formulations are well known and can be found in, for example, Remington's Pharmaceutical Sciences, 18th Ed., Gennaro, ed., Mack Publishing Co., Easton, Pa. 1990.

In various aspects, the disclosed pharmaceutical compositions comprise the disclosed compounds (including pharmaceutically acceptable salt(s) thereof) as an active ingredient, a pharmaceutically acceptable carrier, and, optionally, other therapeutic ingredients or adjuvants. The instant compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

In various aspects, the pharmaceutical compositions of this invention can include a pharmaceutically acceptable carrier and a compound or a pharmaceutically acceptable salt of the compounds of the invention. The compounds of the invention, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques.

A tablet containing the composition of this invention can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

The pharmaceutical compositions of the present invention comprise a compound of the invention (or pharmaceutically acceptable salts thereof) as an active ingredient, a pharmaceutically acceptable carrier, and optionally one or more additional therapeutic agents or adjuvants. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Pharmaceutical compositions of the present invention suitable for parenteral administration can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier canbe a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, mouthwashes, gargles, and the like. Further, the compositions can be in a form suitable for use in transdermal devices. These formulations can be prepared, utilizing a compound of the invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound of the invention, and/or pharmaceutically acceptable salts thereof, can also be prepared in powder or liquid concentrate form.

In a further aspect, an effective amount is a therapeutically effective amount. In a still further aspect, an effective amount is a prophylactically effective amount.

In a further aspect, the pharmaceutical composition is administered to a mammal. In a still further aspect, the mammal is a human. In an even further aspect, the human is a patient.

In a further aspect, the pharmaceutical composition is used to treat a disorder associated with SGLT expression such as, for example, a disorder of uncontrolled cellular proliferation, a neurological disorder, atherosclerosis, diabetes, and heart disease.

It is understood that the disclosed compositions can be prepared from the disclosed compounds. It is also understood that the disclosed compositions can be employed in the disclosed methods of using.

### D. Methods of Making a Compound

The compounds of this invention can be prepared by employing reactions as shown in the following schemes, in addition to other standard manipulations that are known in the literature, exemplified in the experimental sections or clear to one skilled in the art. For clarity, examples having a single substituent are shown where multiple substituents are allowed under the definitions disclosed herein.

Reactions used to generate the compounds of this invention are prepared by employing reactions as shown in the following Reaction Schemes, as described and exemplified below. In certain specific examples, the disclosed compounds can be prepared by Routes I-III, as described and exemplified below. The following examples are provided so that the invention might be more fully understood, are illustrative only, and should not be construed as limiting.

1. Route I

In one aspect, substituted glucose analogs can be prepared as shown below.

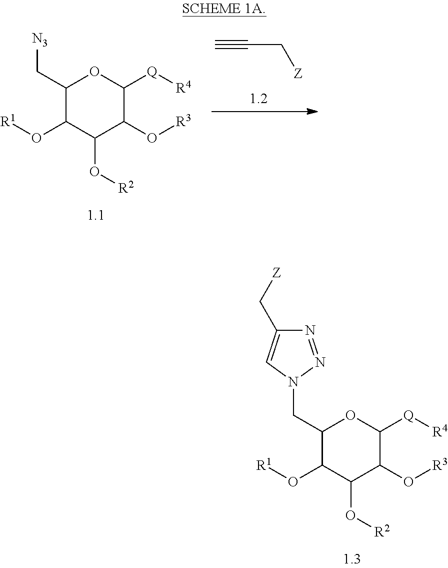

Compounds are represented in generic form, with substituents as noted in compound descriptions elsewhere herein. A more specific example is set forth below.

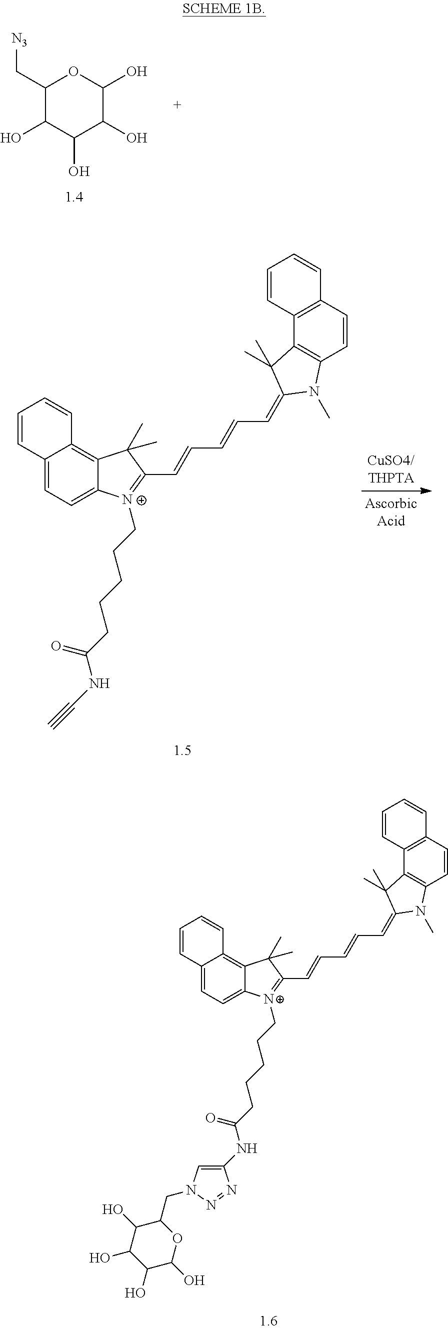 [SMILES: [N-]=[N+]=NCC1OC(O)C(O)C(O)C1O ; C#CNC(=O)CCCCC[N+]1=C(/C=C/C=C/C=C2/N(C)c3ccc4ccccc4c3C2(C)C)C(C)(C)c2c1ccc1ccccc21 ; CN1/C(=C/C=C/C=C/C2=[N+](CCCCCC(=O)Nc3cn(CC4OC(O)C(O)C(O)C4O)nn3)c3ccc4ccccc4c3C2(C)C)C(C)(C)c2c1ccc1ccccc21]

In one aspect, compounds of type 1.6, and similar compounds, can be prepared according to reaction Scheme 1B above. Thus, compounds of type 1.6 can be prepared by a “click” chemistry reaction between an appropriate azide, e.g., 1.4 as shown above, and an appropriate alkyne, e.g., 1.5 as shown above. Appropriate azides and appropriate alkynes are commercially available or prepared by methods known to one skilled in the art. The “click” reaction is carried out in the presence of an appropriate catalyst, e.g., copper (II) sulfate, an appropriate ligand, e.g., tris-hydroxypropyltriazolylmethylamine (THPTA), and an appropriate acid, e.g., ascorbic acid. As can be appreciated by one skilled in the art, the above reaction provides an example of a generalized approach wherein compounds similar in structure to the specific reactants above (compounds similar to compounds of type 1.1 and 1.2), can be substituted in the reaction to provide substituted glucose derivatives similar to Formula 1.3.

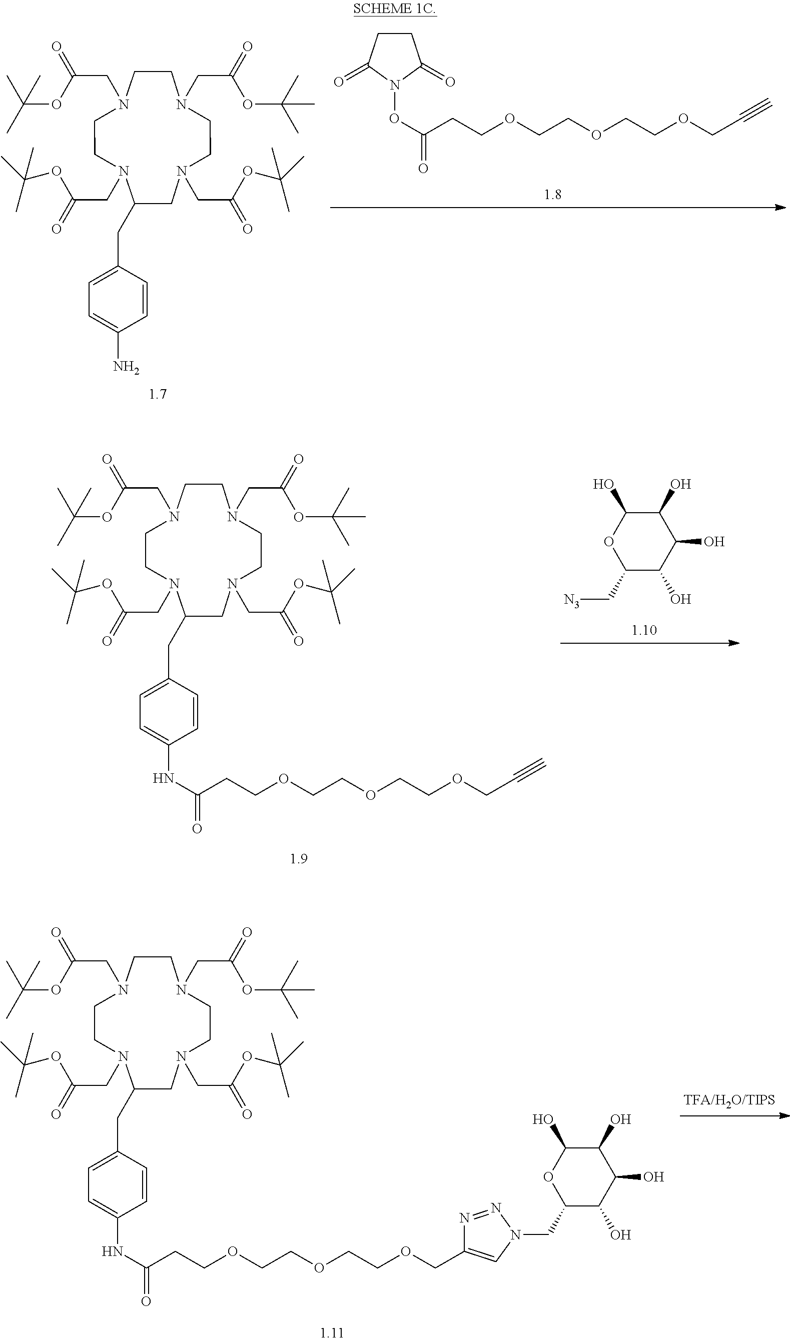

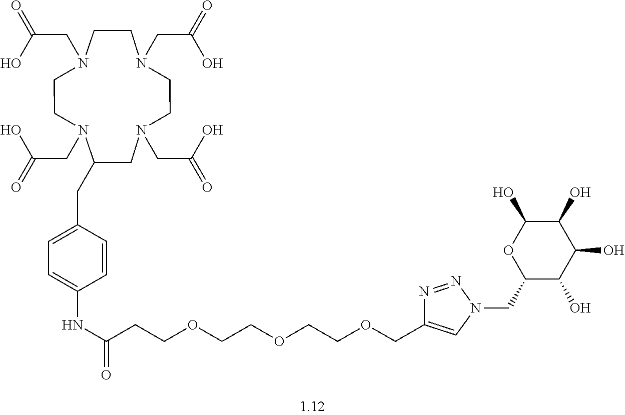 [SMILES: O=C(O)CN1CCN(CC(=O)O)CCN(CC(=O)O)C(Cc2ccc(NC(=O)CCOCCOCCOCc3cn(C[C@@H]4O[C@@H](O)[C@@H](O)[C@@H](O)[C@@H]4O)nn3)cc2)CN(CC(=O)O)CC1]

In one aspect, compounds of type 1.12, and similar compounds, can be prepared according to reaction Scheme 1C above. Thus, compounds of type 1.12 can be prepared by a “click” chemistry reaction between an appropriate azide, e.g., 1.10 as shown above, and an appropriate alkyne, e.g., 1.9 as shown above. Appropriate azides and appropriate alkynes are commercially available or prepared by methods known to one skilled in the art. The “click” reaction is carried out in the presence of an appropriate catalyst, e.g., copper (II) sulfate, an appropriate ligand, e.g., tris-hydroxypropyltriazolylmethylamine (THPTA), and an appropriate acid, e.g., ascorbic acid. As can be appreciated by one skilled in the art, the above reaction provides an example of a generalized approach wherein compounds similar in structure to the specific reactants above (compounds similar to compounds of type 1.1 and 1.2), can be substituted in the reaction to provide substituted glucose derivatives similar to Formula 1.3.

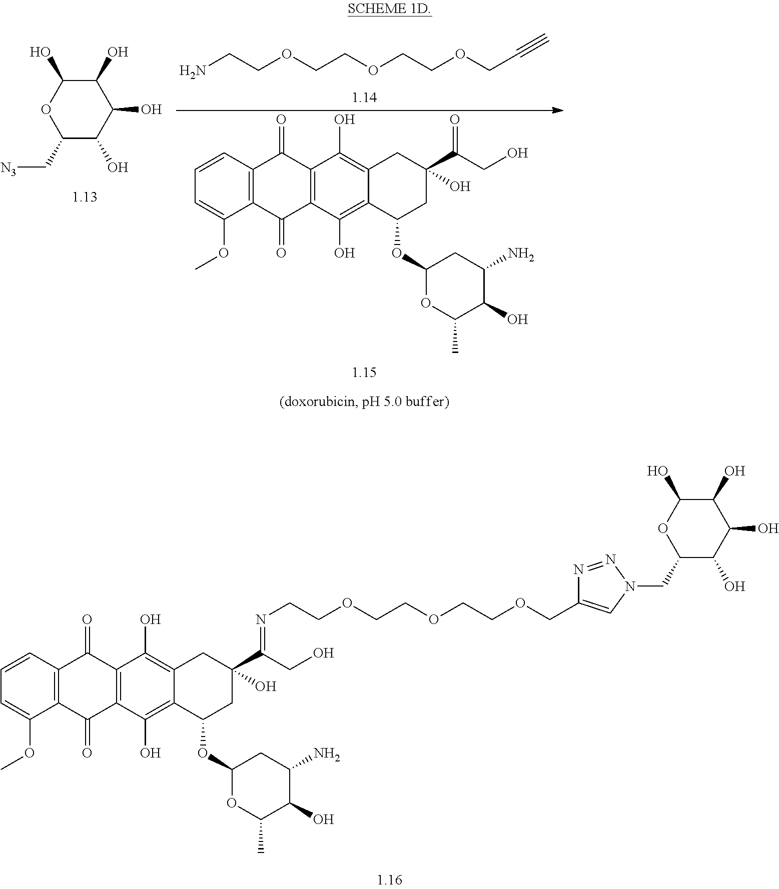 [SMILES: [N-]=[N+]=NC[C@@H]1O[C@@H](O)[C@@H](O)[C@@H](O)[C@@H]1O ; COc1cccc2c1C(=O)c1c(O)c3c(c(O)c1C2=O)C[C@@](O)(C(=O)CO)C[C@@H]3O[C@H]1C[C@H](N)[C@@H](O)[C@H](C)O1 ; C#CCOCCOCCOCCN ; COc1cccc2c1C(=O)c1c(O)c3c(c(O)c1C2=O)C[C@@](O)(/C(CO)=N/CCOCCOCCOCc1cn(C[C@@H]2O[C@@H](O)[C@@H](O)[C@@H](O)[C@@H]2O)nn1)C[C@@H]3O[C@H]1C[C@H](N)[C@@H](O)[C@H](C)O1]

In one aspect, when Z is a therapeutic compound such as a chemotherapeutic (e.g., an anti-cancer antibiotic such as doxorubicin), compounds of type 1.16, and similar compounds, can be prepared according to reaction Scheme 1D above. Thus, compounds of type 1.16 can be prepared by a “click” chemistry reaction between an appropriate azide, e.g., 1.13 as shown above, and an appropriate alkyne, e.g., 1.14 as shown above, in the presence of a suitable therapeutic such as doxorubicin (1.15 as shown above). Appropriate azides and appropriate alkynes are commercially available or prepared by methods known to one skilled in the art. The “click” reaction is carried out in the presence of an appropriate catalyst, e.g., copper (II) sulfate, an appropriate ligand, e.g., tris-hydroxypropyltriazolylmethylamine (THPTA), and an appropriate acid, e.g., ascorbic acid. As can be appreciated by one skilled in the art, the above reaction provides an example of a generalized approach wherein compounds similar in structure to the specific reactants above (compounds similar to compounds of type 1.1 and 1.2), can be substituted in the reaction to provide substituted glucose derivatives similar to Formula 1.3.

2. Route II

In one aspect, substituted glucose analogs can be prepared as shown below.

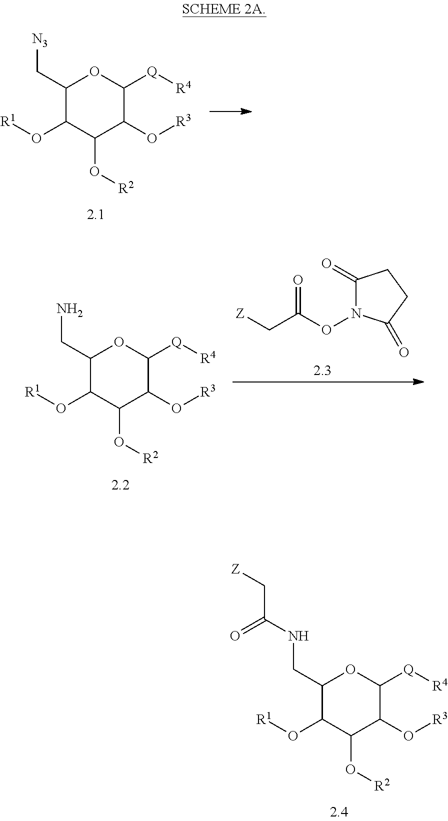

Compounds are represented in generic form, with substituents as noted in compound descriptions elsewhere herein. A more specific example is set forth below.

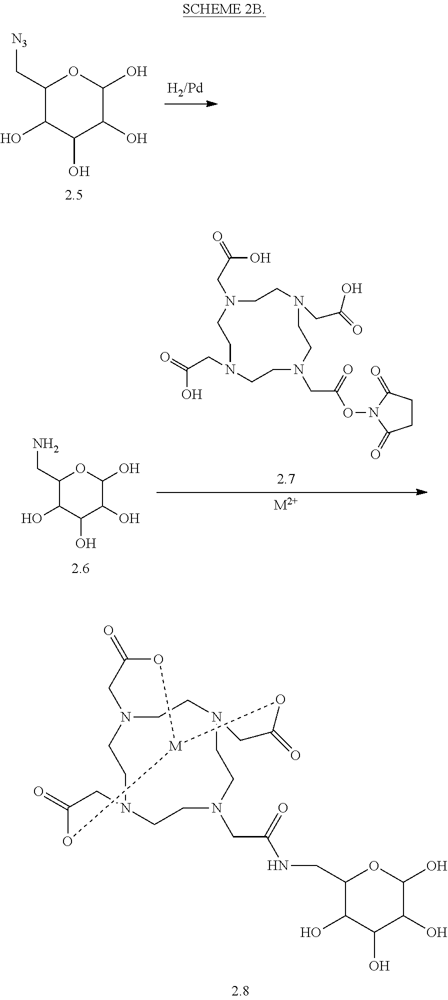

In one aspect, compounds of type 2.8, and similar compounds, can be prepared according to reaction Scheme 2B above. Thus, compounds of type 2.6 can be prepared by reduction of an appropriate azie, e.g., 2.5 as shown above. Appropriate azides are commercially available or prepared by methods known to one skilled in the art. The reduction is carried out in the presence of an appropriate reducing agent, e.g., hydrogen gas, and an appropriate catalyst, e.g., palladium. Compounds of type 2.8 can be prepared by coupling an appropriate amine, e.g., 2.6 as shown above, with an appropriate activated acid, e.g., 2.7 as shown above. The coupling reaction is carried out in the presence of an appropriate radioisotope, M² as shown above. As can be appreciated by one skilled in the art, the above reaction provides an example of a generalized approach wherein compounds similar in structure to the specific reactants above (compounds similar to compounds of type 2.1, 2.2, and 2.3), can be substituted in the reaction to provide substituted glucose derivatives similar to Formula 2.4.

3. Route III

In one aspect, substituted glucose analogs can be prepared as shown below.

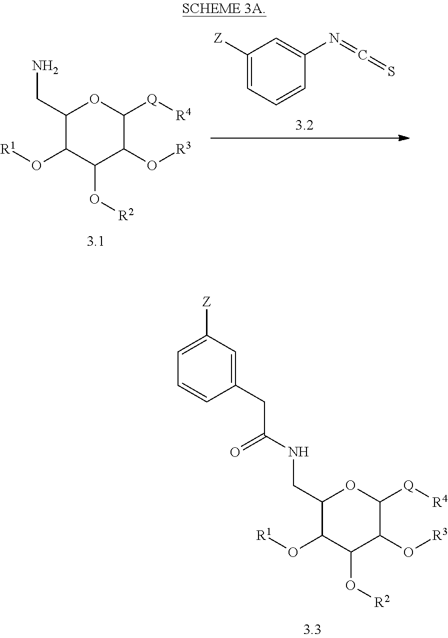

Compounds are represented in generic form, with substituents as noted in compound descriptions elsewhere herein. A more specific example is set forth below.

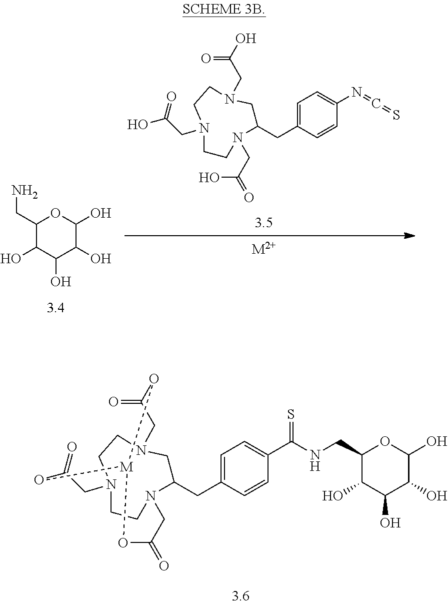

In one aspect, compounds of type 1.6, and similar compounds, can be prepared according to reaction Scheme 3B above. Thus, compounds of type 3.6 can be prepared by a coupling reaction between an appropriate amine, e.g., 3.4 as shown above, and an appropriate isothiocyanate, e.g., 3.5 as shown above. Appropriate amines and appropriate isothiocyanates are commercially available or prepared by methods known to one skilled in the art. The coupling reaction is carried out in the presence of an appropriate radioisotope, M² as shown above. As can be appreciated by one skilled in the art, the above reaction provides an example of a generalized approach wherein compounds similar in structure to the specific reactants above (compounds similar to compounds of type 3.1 and 3.2), can be substituted in the reaction to provide substituted glucose derivatives similar to Formula 3.3.

### E. Methods of Using the Compounds

The compounds and pharmaceutical compositions of the invention are useful in treating or controlling disorders associated with SGLT expression, in particular, disorders of uncontrolled cellular proliferation, neurological disorders, atherosclerosis, diabetes, and heart disease.

In various aspects, the disclosed compounds and pharmaceutical compositions are useful in treating or controlling disorders of uncontrolled cellular proliferation. In a further aspect, the disorder of uncontrolled cellular proliferation is a cancer. Examples of cancers for which the compounds and compositions can be useful in treating, include, but are not limited to, sarcomas, carcinomas, hematological cancers, solid tumors, breast cancer, cervical cancer, gastrointestinal cancer, colorectal cancer, brain cancer, skin cancer, prostate cancer, ovarian cancer, thyroid cancer, testicular cancer, pancreatic cancer, endometrial cancer, melanomas, gliomas, leukemia, lymphoma, chronic myeloproliferative disorder, myelodysplastic syndrome, myeloproliferative neoplasm, and plasma cell neoplasm (myeloma). In a still further aspect, the cancer is a primary tumor or a mestastes. In yet a further aspect, the cancer is selected from colorectal cancer, pancreatic cancer, ovarian cancer, breast cancer, head and neck cancer, prostate cancer, and lung cancer.

In various aspects, the disclosed compounds and pharmaceutical compositions are useful in treating or controlling neurological disorders. Examples of neurological disorders include, but are not limited to, amytrophic lateral sclerosis, arteriovenous malformation, brain aneurysms, brain tumors, dural arteriovenous fistulae, epilepsy, headaches, memory disorders, multiple sclerosis, Parkinson's disease, peripheral neuropathy, post-herpetic neuralgia, spinal cord tumors, and stroke.

In various aspects, the disclosed compounds and pharmaceutical compositions are useful in treating or controlling atherosclerosis.

In various aspects, the disclosed compounds and pharmaceutical compositions are useful in treating or controlling diabetes.

In various aspects, the disclosed compounds and pharmaceutical compositions are useful in treating or controlling heart disease.

To treat or control the disorder, the compounds and pharmaceutical compositions comprising the compounds are administered to a subject in need thereof, such as a vertebrate, e.g., a mammal, a fish, a bird, a reptile, or an amphibian. The subject can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. The subject is preferably a mammal, such as a human. Prior to administering the compounds or compositions, the subject can be diagnosed with a need for treatment of a disorder associated with SGLT expression, such as, for example, disorders of uncontrolled cellular proliferation, neurological disorders, atherosclerosis, diabetes, and heart disease.

The compounds or compositions can be administered to the subject according to any method. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration and parenteral administration, including injectable such as intraperitoneal administration, intravenous administration, intra-arterial administration, intramuscular administration, and subcutaneous administration.

Administration can be continuous or intermittent. A preparation can be administered therapeutically; that is, administered to treat an existing disease or condition. A preparation can also be administered prophylactically; that is, administered for prevention of a disorder associated with SGLT expression, such as, for example, disorders of uncontrolled cellular proliferation, neurological disorders, atherosclerosis, diabetes, and heart disease.

The therapeutically effective amount or dosage of the compound can vary within wide limits. Such a dosage is adjusted to the individual requirements in each particular case including the specific compound(s) being administered, the route of administration, the condition being treated, as well as the patient being treated. In general, in the case of oral or parenteral administration to adult humans weighing approximately 70 Kg or more or less, a daily dosage of about 10 mg to about 10,000 mg, preferably from about 200 mg to about 1,000 mg, should be appropriate, although the upper limit may be exceeded. The daily dosage can be administered as a single dose or in divided doses, or for parenteral administration, as a continuous infusion. Single dose compositions can contain such amounts or submultiples thereof of the compound or composition to make up the daily dose. The dosage can be adjusted by the individual physician in the event of any contraindications. Dosage can vary, and can be administered in one or more dose administrations daily, for one or several days.

1. Treatment Methods

The compounds disclosed herein are useful for treating or controlling disorders associated with SGLT expression, such as, for example, disorders of uncontrolled cellular proliferation, neurological disorders, atherosclerosis, diabetes, and heart disease. Thus, provided is a method comprising administering a therapeutically effective amount of a composition comprising a disclosed compound to a subject. In a further aspect, the method can be a method for treating a viral infection.

a. Treating a Disorder Associated with Sglt Expression

In one aspect, disclosed are methods of treating a disorder associated with SGLT expression in a subject, the method comprising the step of administering to the subject a therapeutically effective amount of at least one disclosed compound, or a pharmaceutically acceptable salt thereof.

In one aspect, disclosed are methods for the treatment of a disorder associated with SGLT expression in a subject, the method comprising the step of administering to the subject a therapeutically effective amount of at least one compound having a structure represented by a formula:

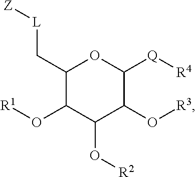

wherein L is a divalent linker; wherein Q is selected from —O—, —NHC(O)—, and —NHC(S)NH—; wherein Z is a fluorophore, a radiolabel, a radioisotope, or a radiotherapeutic; and wherein each of R¹, R², R³, and R⁴ is independently selected from hydrogen, C1-C4 alkyl, aryl, a fluorophore residue, a peptide residue, and a polyaromatic residue, provided that when Q is —O— and Z is a fluorophore, then at least one of R¹, R², R³, and R⁴ is not hydrogen, or a pharmaceutically acceptable salt thereof, wherein the disorder is a disorder of uncontrolled cellular proliferation, a neurological disorder, atherosclerosis, diabetes, or heart disease.

In various aspects, the disorder is a disorder of uncontrolled cellular proliferation. In a further aspect, the disorder of uncontrolled cellular proliferation is a cancer. Examples of cancers for which the compounds and compositions can be useful in treating, include, but are not limited to, sarcomas, carcinomas, hematological cancers, solid tumors, breast cancer, cervical cancer, gastrointestinal cancer, colorectal cancer, brain cancer, skin cancer, prostate cancer, ovarian cancer, thyroid cancer, testicular cancer, pancreatic cancer, endometrial cancer, melanomas, gliomas, leukemia, lymphoma, chronic myeloproliferative disorder, myelodysplastic syndrome, myeloproliferative neoplasm, and plasma cell neoplasm (myeloma). In a still further aspect, the cancer is a primary tumor or a mestastes. In yet a further aspect, the cancer is selected from colorectal cancer, pancreatic cancer, ovarian cancer, breast cancer, head and neck cancer, prostate cancer, and lung cancer/

In various aspects, the disorder is a neurological disorder. Examples of neurological disorders include, but are not limited to, amytrophic lateral sclerosis, arteriovenous malformation, brain aneurysms, brain tumors, dural arteriovenous fistulae, epilepsy, headaches, memory disorders, multiple sclerosis, Parkinson's disease, peripheral neuropathy, post-herpetic neuralgia, spinal cord tumors, and stroke.

In various aspects, the disorder is atherosclerosis.

In various aspects, the disorder is diabetes.

In various aspects, the disorder is heart disease.

In a further aspect, the subject has been diagnosed with a need for treatment of the disorder prior to the administering step.

In a further aspect, the subject is a mammal. In a still further aspect, the mammal is a human.

In a further aspect, the method further comprises the step of identifying a subject in need of treatment of the disorder.

In a further aspect, the effective amount is a therapeutically effective amount.

In a still further aspect, the effective amount is a prophylactically effective amount.

2. Methods of Modifying Sglt Expression in a Subject

In one aspect, disclosed are methods of modifying SGLT expression in a subject, the method comprising the step of administering to the subject a therapeutically effective amount of at least one disclosed compound, or a pharmaceutically acceptable salt thereof.

Thus, in one aspect, disclosed are methods of modifying SGLT expression in a subject, the method comprising the step of administering to the subject a therapeutically effective amount of at least one compound having a structure represented by a formula:

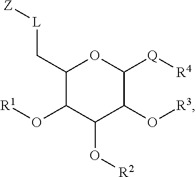

wherein L is a divalent linker; wherein Q is selected from —O—, —NHC(O)—, and —NHC(S)NH—; wherein Z is a fluorophore, a radiolabel, a radioisotope, or a radiotherapeutic; and wherein each of R¹, R², R³, and R⁴ is independently selected from hydrogen, C1-C4 alkyl, aryl, a fluorophore residue, a peptide residue, and a polyaromatic residue, provided that when Q is —O— and Z is a fluorophore, then at least one of R¹, R², R³, and R⁴ is not hydrogen, or a pharmaceutically acceptable salt thereof.

In a further aspect, the compound exhibits inhibition of SGLT expression.

In a further aspect, the compound exhibits knock-down or knock-up of SGLT expression. In a still further aspect, the compound exhibits knock-down of SGLT expression.

In yet a further aspect, the compound exhibits knock-up of SGLT expression.

In a further aspect, the subject is a mammal. In a still further aspect, the subject is a human.

In a further aspect, the subject has been diagnosed with a need for modifying SGLT expression prior to the administering step.

In a further aspect, the subject has been diagnosed with a need for treatment of a disorder associated with SGLT expression prior to the administering step. In a still further aspect, the disorder associated with SGLT expression is a disorder of uncontrolled cellular proliferation, diabetes, heart disease, or a neurological disorder. In yet a further aspect, the method further comprises the step of identifying a subject in need of treatment of a disorder associated with dysfunction of SGLT expression.

3. Methods of Modifying Sglt Expression in at Least One Cell

In one aspect, disclosed are methods for modifying SGLT expression in at least one cell, the method comprising the step of contacting the at least one cell with an effective amount of at least one disclosed compound, or a pharmaceutically acceptable salt thereof.

Thus, in one aspect, disclosed are methods for modifying SGLT expression in at least one cell, the method comprising the step of contacting the at least one cell with an effective amount of at least one compound having a structure represented by a formula:

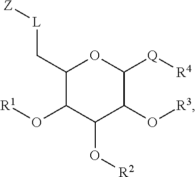

wherein L is a divalent linker; wherein Q is selected from —O—, —NHC(O)—, and —NHC(S)NH—; wherein Z is a fluorophore, a radiolabel, a radioisotope, or a radiotherapeutic; and wherein each of R¹, R², R³, and R⁴ is independently selected from hydrogen, C1-C4 alkyl, aryl, a fluorophore residue, a peptide residue, and a polyaromatic residue, provided that when Q is —O— and Z is a fluorophore, then at least one of R, R², R³, and R⁴ is not hydrogen, or a pharmaceutically acceptable salt thereof.

In a further aspect, the compound exhibits inhibition of SGLT expression.

In a further aspect, the compound exhibits knock-down or knock-up of SGLT expression. In a still further aspect, the compound exhibits knock-down of SGLT expression.

In yet a further aspect, the compound exhibits knock-up of SGLT expression.

In a further aspect, the cell is mammalian. In a still further aspect, the cell is human. In yet a further aspect, the cell has been isolated from a mammal prior to the contacting step.

In a further aspect, contacting is via administration to a mammal.

In a further aspect, the subject has been diagnosed with a need for modification of SGLT expression prior to the administering step. In a still further aspect, the subject has been diagnosed with a need for treatment of a disorder associated with dysfunction of SGLT expression.

4. Use of Compounds

In one aspect, the invention relates to the use of a disclosed compound or a product of a disclosed method. In a further aspect, a use relates to the manufacture of a medicament for the treatment of a disorder associated with SGLT expression in a subject. In a still further aspect, the disorder is a disorder of uncontrolled cellular proliferation, a neurological disorder, atherosclerosis, diabetes, or heart disease.

Also provided are the uses of the disclosed compounds and products. In one aspect, the invention relates to use of at least one disclosed compound; or a pharmaceutically acceptable salt, hydrate, solvate, or polymorph thereof. In a further aspect, the compound used is a product of a disclosed method of making.

In a further aspect, the use relates to a process for preparing a pharmaceutical composition comprising a therapeutically effective amount of a disclosed compound or a product of a disclosed method of making, or a pharmaceutically acceptable salt, solvate, or polymorph thereof, for use as a medicament.

In a further aspect, the use relates to a process for preparing a pharmaceutical composition comprising a therapeutically effective amount of a disclosed compound or a product of a disclosed method of making, or a pharmaceutically acceptable salt, solvate, or polymorph thereof, wherein a pharmaceutically acceptable carrier is intimately mixed with a therapeutically effective amount of the compound or the product of a disclosed method of making.

In various aspects, the use relates to a treatment of a disorder associated with SGLT expression in a subject. Also disclosed is the use of a compound for inhibition of a SGLT expression. Also disclosed is the use of a compound for knock-down or knock-up of SGLT expression.

In one aspect, the use is characterized in that the subject is a human. In one aspect, the use is characterized in that the disorder is a disorder associated with SGLT expression such as, for example, a disorder of uncontrolled cellular proliferation, a neurological disorder, atherosclerosis, diabetes, or heart disease.

In a further aspect, the use relates to the manufacture of a medicament for the treatment of a disorder associated with SGLT expression (e.g., a disorder of uncontrolled cellular proliferation, a neurological disorder, atherosclerosis, diabetes, or heart disease) in a subject.

In a further aspect, the use relates to modulating SGLT expression in a subject.

In a still further aspect, the use relates to modulating SGLT expression in a cell. In yet a further aspect, the subject is a human.

It is understood that the disclosed uses can be employed in connection with the disclosed compounds, products of disclosed methods of making, methods, compositions, and kits. In a further aspect, the invention relates to the use of a disclosed compound or a disclosed product in the manufacture of a medicament for the treatment of a disorder associated with SGLT expression. In a still further aspect, the disorder is a disorder of uncontrolled cellular proliferation, a neurological disorder, atherosclerosis, diabetes, or heart disease.

5. Manufacture of a Medicament

In one aspect, the invention relates to a method for the manufacture of a medicament for treating a disorder associated with SGLT expression in a subject, the method comprising combining a therapeutically effective amount of a disclosed compound or product of a disclosed method with a pharmaceutically acceptable carrier or diluent.

As regards these applications, the present method includes the administration to an animal, particularly a mammal, and more particularly a human, of a therapeutically effective amount of the compound effective in the treatment of a disorder associated with SGLT expression. The dose administered to an animal, particularly a human, in the context of the present invention should be sufficient to affect a therapeutic response in the animal over a reasonable time frame. One skilled in the art will recognize that dosage will depend upon a variety of factors including the condition of the animal and the body weight of the animal.

The total amount of the compound of the present disclosure administered in a typical treatment is preferably between about 10 mg/kg and about 1000 mg/kg of body weight for mice, and between about 100 mg/kg and about 500 mg/kg of body weight, and more preferably between 200 mg/kg and about 400 mg/kg of body weight for humans per daily dose. This total amount is typically, but not necessarily, administered as a series of smaller doses over a period of about one time per day to about three times per day for about 24 months, and preferably over a period of twice per day for about 12 months.

The size of the dose also will be determined by the route, timing and frequency of administration as well as the existence, nature and extent of any adverse side effects that might accompany the administration of the compound and the desired physiological effect. It will be appreciated by one of skill in the art that various conditions or disease states, in particular chronic conditions or disease states, may require prolonged treatment involving multiple administrations.

Thus, in one aspect, the invention relates to the manufacture of a medicament comprising combining a disclosed compound or a product of a disclosed method of making, or a pharmaceutically acceptable salt, solvate, or polymorph thereof, with a pharmaceutically acceptable carrier or diluent.

6. Kits

In one aspect, disclosed are kits comprising comprising at least one disclosed compound, and one or more of: (a) at least one agent associated with the treatment of a disorder associated with SGLT expression, wherein the disorder is selected from a disorder of uncontrolled cellular proliferation, a neurological disorder, atherosclerosis, diabetes, and heart disease; (b) instructions for administering the compound in connection with treating the disorder; and (c) instructions for treating the disorder.

In one aspect, disclosed are kits comprising comprising at least one compound having a structure represented by a formula:

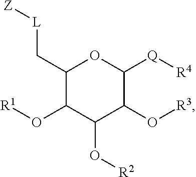

wherein L is a divalent linker; wherein Q is selected from —O—, —NHC(O)—, and —NHC(S)NH—; wherein Z is a fluorophore, a radiolabel, a radioisotope, or a radiotherapeutic; and wherein each of R¹, R², R³, and R⁴ is independently selected from hydrogen, C1-C4 alkyl, aryl, a fluorophore residue, a peptide residue, and a polyaromatic residue, provided that when Q is —O— and Z is a fluorophore, then at least one of R¹, R², R³, and R⁴ is not hydrogen, or a pharmaceutically acceptable salt thereof, and one or more of: (a) at least one agent associated with the treatment of a disorder associated with SGLT expression, wherein the disorder is selected from a disorder of uncontrolled cellular proliferation, a neurological disorder, atherosclerosis, diabetes, and heart disease; (b) instructions for administering the compound in connection with treating the disorder; and (c) instructions for treating the disorder.

In a further aspect, the agent is a chemotherapeutic agent. In a still further aspect, the chemotherapeutic agent is selected from an alkylating agent, an antimetabolite agent, an antineoplastic antibiotic agent, a mitotic inhibitor agent, a tyrosine kinase inhibitor (TKI), a poly ADP ribose polymerase (PARP) inhibitor, and a mTor inhibitor agent.

In a further aspect, the chemotherapeutic agent is an antineoplastic antibiotic agent. Examples of antineoplastic antibiotic agents include, but are not limited to, doxorubicin, mitoxantrone, bleomycin, daunorubicin, dactinomycin, epirubicin, idarubicin, plicamycin, mitomycin, pentostatin, and valrubicin, or a pharmaceutically acceptable salt thereof.

In a further aspect, the chemotherapeutic agent is an antimetabolite agent. Examples of antimetabolite agents include, but are not limited to, gemcitabine, 5-fluorouracil, capecitabine, hydroxyurea, mercaptopurine, pemetrexed, fludarabine, nelarabine, cladribine, clofarabine, cytarabine, decitabine, pralatrexate, floxuridine, methotrexate, and thioguanine, or a pharmaceutically acceptable salt thereof.

In a further aspect, the chemotherapeutic agent is an alkylating agent. Examples of alkylating agents include, but are not limited to, carboplatin, cisplatin, cyclophosphamide, chlorambucil, melphalan, carmustine, busulfan, lomustine, dacarbazine, oxaliplatin, ifosfamide, mechlorethamine, temozolomide, thiotepa, bendamustine, and streptozocin, or a pharmaceutically acceptable salt thereof.

In a further aspect, the chemotherapeutic agent is a mitotic inhibitor agent. Examples of mitotic inhibitor agents include, but are not limited to, irinotecan, topotecan, rubitecan, cabazitaxel, docetaxel, paclitaxel, etoposide, vincristine, ixabepilone, vinorelbine, vinblastine, and teniposide, or a pharmaceutically acceptable salt thereof.

In a further aspect, the chemotherapeutic agent is a mTor inhibitor agent. Examples of mTor inhibitor agent include, but are not limited to, everolimus, siroliumus, and temsirolimus, or a pharmaceutically acceptable salt, hydrate, solvate, or polymorph thereof.

In a further aspect, the at least one compound and the at least one agent are co-formulated. In a further aspect, the at least one compound and the at least one agent are co-packaged.

In various aspects, the disorder is a disorder of uncontrolled cellular proliferation. In a further aspect, the disorder of uncontrolled cellular proliferation is a cancer. Examples of cancers for which the compounds and compositions can be useful in treating, include, but are not limited to, sarcomas, carcinomas, hematological cancers, solid tumors, breast cancer, cervical cancer, gastrointestinal cancer, colorectal cancer, brain cancer, skin cancer, prostate cancer, ovarian cancer, thyroid cancer, testicular cancer, pancreatic cancer, endometrial cancer, melanomas, gliomas, leukemia, lymphoma, chronic myeloproliferative disorder, myelodysplastic syndrome, myeloproliferative neoplasm, and plasma cell neoplasm (myeloma). In a still further aspect, the cancer is a primary tumor or a mestastes. In yet a further aspect, the cancer is selected from colorectal cancer, pancreatic cancer, ovarian cancer, breast cancer, head and neck cancer, prostate cancer, and lung cancer/

In various aspects, the disorder is a neurological disorder. Examples of neurological disorders include, but are not limited to, amytrophic lateral sclerosis, arteriovenous malformation, brain aneurysms, brain tumors, dural arteriovenous fistulae, epilepsy, headaches, memory disorders, multiple sclerosis, Parkinson's disease, peripheral neuropathy, post-herpetic neuralgia, spinal cord tumors, and stroke.

In various aspects, the disorder is atherosclerosis.

In various aspects, the disorder is diabetes.

In various aspects, the disorder is heart disease.

The kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

It is understood that the disclosed kits can be prepared from the disclosed compounds, products, and pharmaceutical compositions. It is also understood that the disclosed kits can be employed in connection with the disclosed methods of using.

The foregoing description illustrates and describes the disclosure. Additionally, the disclosure shows and describes only the preferred embodiments but, as mentioned above, it is to be understood that it is capable to use in various other combinations, modifications, and environments and is capable of changes or modifications within the scope of the invention concepts as expressed herein, commensurate with the above teachings and/or the skill or knowledge of the relevant art. The embodiments described herein above are further intended to explain best modes known by applicant and to enable others skilled in the art to utilize the disclosure in such, or other, embodiments and with the various modifications required by the particular applications or uses thereof. Accordingly, the description is not intended to limit the invention to the form disclosed herein. Also, it is intended to the appended claims be construed to include alternative embodiments.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

### F. Examples

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

The Examples are provided herein to illustrate the invention, and should not be construed as limiting the invention in any way. Examples are provided herein to illustrate the invention and should not be construed as limiting the invention in any way.

1. Exploring Sglts as a Theranostic Target in Cancer Metastasis

SGLT1 is overexpressed in prostate, breast, cervical, colorectal, head and neck, pancreatic, and lung cancers (Blessing et al. (2012) J. Cancer Sci Ther. 4: 306-12; Weihua et al. (2008) Cancer Cell 13: 385-93; Huang et al. (2012) Physiological measurement 33:1661-73; Perez et al. (2013) PloS one 8:e56169). SGLT1 is only weakly expressed in normal prostate epithelium and universally overexpressed in prostate cancer with differential patterns of expression between low and high grade cancers (Blessing et al. (2012) J. Cancer Sci Ther. 4: 306-12). SGLT2 is overexpressed in breast, prostate, colorectal, gastrointestinal, head and neck, and renal carcinomas as well as some leukemias (Scafoglio et al. (2015) Proceedings of the National Academy of Sciences of the United States ofAmerica 112:E4111-9; Cao et al. (2007) Cancer chemotherapy andpharmacology 59:495-505; Szablewski L. (2013) Biochimica et biophysica acta 1835:164-9). Further, early clinical studies suggest that the SGLT family of glucose transporters may potentially serve as important prognostic biomarkers in select cancers. For example, Casneuf et al. studied the expression of SGLT1 in primary adenocarcinomas of the pancreas, and multivariate analysis demonstrated that SGLT1 expression was prognostic for greater disease free survival (DFS) with a trend for greater overall survival (OS) (Casneuf et al. (2008) Cancer investigation 26:852-9) whereas Scafoglio et al. have shown SGLT2 is functionally expressed in pancreatic adenocarcinoma and that its inhibitors block glucose uptake and reduce tumor growth (Scafoglio et al. (2015) Proceedings of the National Academy of Sciences of the United States of America 112:E4111-9). In a clinical study involving 85 patients with colorectal cancer receiving first-line chemotherapy, Guo et al. found expression of SGLT1 correlated with clinical stage and demonstrated a trend (p=0.06) for predicting clinical response rate (Guo et al. (2011) Med Oncol. 28 Suppl 1:S197-203). Similar results have also been seen in ovarian carcinomas where SGLT1 overexpression correlated with clinical stage, and further, SGLT1 protein expression was an independent prognostic predictor for patient survival (Lai et al. (2012) Archives of gynecology and obstetrics 285:1455-61). Taken together, these works suggest the SGLTs could serve as an important clinical target for diagnosis and therapy.

Herein, a treatment paradigm that may prove to be more effective and less toxic than existing therapies, and even succeed where targeted therapies have failed, is developed. As stated above, for normal and especially cancer cells, glucose transporters are essential as the rate of glucose diffusion across the plasma membrane is not sufficient for cell survival, growth, and proliferation. SGLTs may be particularly critical for survival in ischemic tumor microenvironments, such as cancer metastasis, which have much lower glucose concentrations than corresponding normal tissues (Huber et al. (2012) Radiotherapy and oncology: journal of the European Society for Therapeutic Radiology and Oncology 103:373-9; Urasaki et al. (2012) PloS one 7:e36775); SGLTs enable concentration of glucose by coupling the transport of glucose to that of sodium flowing down its electrochemical gradient and, thus, unlike GLUTs, can concentrate glucose intracellularly up to 400 times (SGLT1) its extracellular concentration (Kimmich and Randles (1982) The American journal of physiology 241: 227-32). Various reports have shown that SGLT expression is even higher in metastatic than in primary tumors, and contributes to enhanced chemoresistance of metastasis (Szablewski L. (2013) Biochimica et biophysica acta 1835:164-9), which further supports the selection of SGLT as a target. Further, contemporary targeted therapies treat tumors as a single clonal population and do not consider tumor heterogeneity. For example, targeted therapies (e.g., kinase inhibitors) can only eradicate cancer cells with the protein mutation or amplification, and variation in the cancer cell population can lead to disease persistence and recurrence. Thus, a theranostic approach is proposed that combines SGLT targeting with radionuclide therapy (⁶⁸Ga and ¹⁷⁷Lu, for example), optionally in combination with another therapeutic, which provides a unique opportunity to eradicate metastatic disease. SGLTs, which are increased in the ischemic tumor microenvironment, will concentrate the therapeutic agent in SGLT-expressing cancer cells killing them. Significantly, adjacent cancer cells that do not express SGLTs can also be eradicated due to “cross-fire” (radiation deposition in nearby cells that are not specifically targeted) to a distance of many cell lengths from SGLT-expressing cells (Ji et al. (2014) Oncotarget. 5:2934-46; Shukla et al. (2012) Proceedings of the National Academy of Sciences of the United States of America 109:12426-31; Ersahin et al. (2011) Cancers 3:3838-55). This tackles two important challenges for systemic therapy: (1) delivery within the hostile tumor microenvironment; and (2) overcoming tumor heterogeneity. It particularly addresses the challenge of triple-negative breast cancer where even adjacent cells are heterogeneous making conventional targeted therapy unlikely to succeed (Huebschman et al. (2015) Breast Cancer (Dove Med Press) 7:231-7). The targeted radionuclide therapy approach has been previously translated to the clinic with commercially-available agents such as ²²³RaCl₂ (Xofigo®), [¹³¹I]tositumomab (Bexxar®), and [⁹⁰Y]ibritumomab tiuxetan (Zevalin®). Further, in a landmark randomized phase III clinical trial, Xofigo, a ²²³Ra alpha emitter, was shown to improve overall survival in men with metastatic prostate cancer, and, importantly, low rates of myelosuppression and few adverse events (Parker et al. (2013) The New Englandjournal of medicine 369:213-23). However, despite its success, Xofigo has limitations such as it only targets bone metastases and the lack of imaging prohibits patient-specific treatment planning. For reasons of economy and efficiency, exploratory and preliminary studies will be completed using a near-infrared fluorophore (NIRF) label with the intent of translation to clinical relevant radiolabels in future studies. Importantly, SGLT selectivity should avoid neurotoxic effects associated with accumulation in the brain as the blood-brain barrier (BBB) expresses GLUT1; SGLT-selective compounds can only enter brain if BBB is disrupted.

2. Development of SGLTs

Herein, it is proposed to image SGLTs to assess their potential as a theranostic target for cancer. Conventional PET oncologic imaging uses [¹⁸F]FDG, which is well-transported by GLUTs but poorly by SGLTs (Landau et al. (2007) American journal of physiology Endocrinology and metabolism 293:E237-45; Yu et al. (2010) American journal of physiology Cell physiology 299:C1277-84). This focus on SGLTs differs from conventional PET approaches and is consistent with emerging literature on glucose transport in cancer. SGLTs, unlike GLUTs, can concentrate glucose inside the cell, even when the interstitial glucose concentration is low. With SGLTs, intracellular glucose concentration is powered by coupling transport of glucose or an analog to that of the sodium ion. This is profoundly different from the two-step process that is the basis conventional [¹⁸F]FDG-PET: GLUT-facilitated transport down a concentration gradient followed by phosphorylation by hexokinase to achieve intracellular entrapment. For this reason, using SGLTs as an theranostic is distinct from conventional [¹⁸F]FDG-PET (GLUTs and hexokinase) imaging, revealing cancer metabolism that is supported by SGLTs and not GLUTs. Moreover, the ability to concentrate glucose analogs from 10 to 400-fold intracellularly (Kimmich and Randles (1981) The American journal of physiology 241:C227-32) is expected to achieve a local therapeutic dose using only a low systemic concentration thereby minimizing toxicities. Further, this is more than sufficient for imaging; in routine [¹⁸F]FDG-PET scanning, three-fold concentrating (Specific Uptake Value, SUV=3 g/ml) is readily visualized.

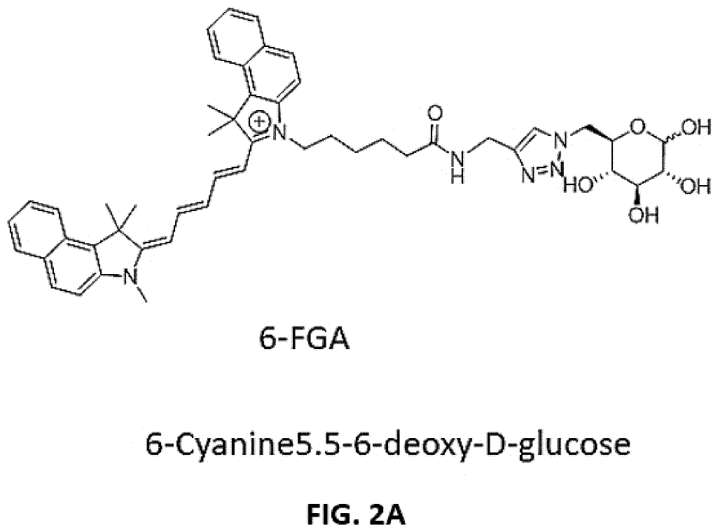

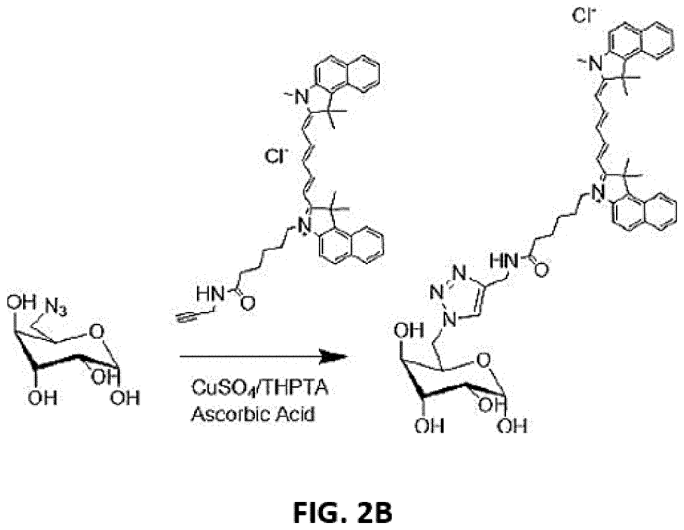

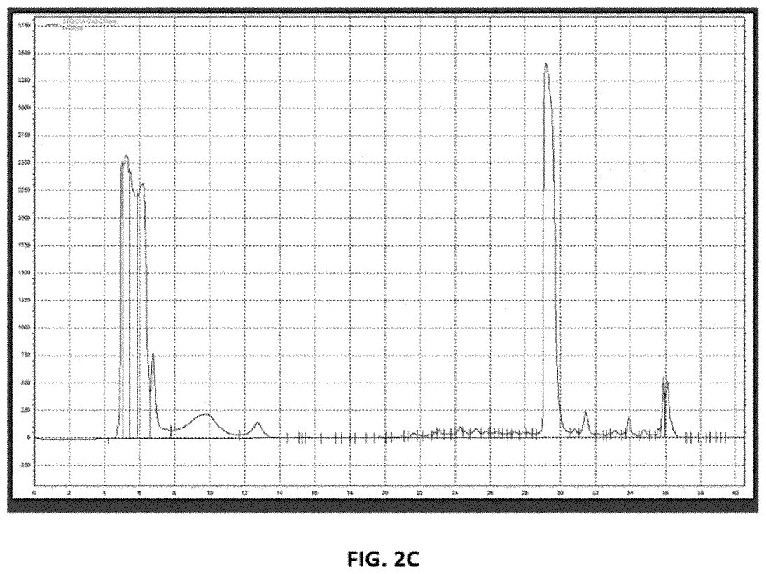

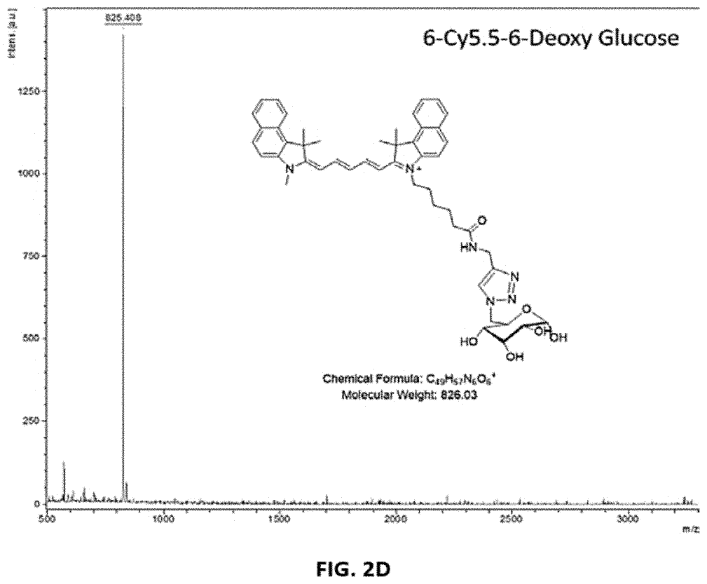

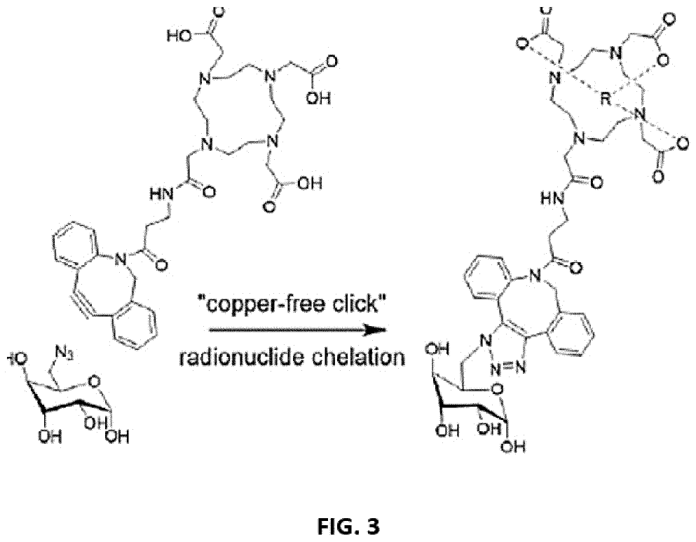

Based on prior work with [¹⁸F]6FDG [R01 DK14507, DK082423] (Landau et al. (2007) American journal of physiology Endocrinology and metabolism 293: E237-45; Muzic et al. (2011) Nuclear medicine and biology 38:667-74; Neal et al. (2005) J Labelled Compd Rad. 48:845-54; Spring-Robinson (2009) Journal of nuclear medicine: official publication, Society of Nuclear Medicine 50:912-9; Huang et al. (2012) Physiological measurement 33:1661-73; Muzic et al. (2014) Medical physics. 41:031910; Su et al. (2014) Molecular imaging and biology: MIB: the official publication of the Academy of Molecular Imaging. 16:710-20), it is proposed that a C6-modified glucose analog would allow SGLT-selectivity and would not be phosphorylatable and subsequently metabolized due to the absence of the hydroxyl group on C6 as is the case with glucose. For reasons of economy and efficiency, which are needed for translational exploratory/developmental studies (PAR-18-020), a fluorophore was used for the label. Specifically, click chemistry between a 6-azido-D-glucose and a Near Infrared fluorophore (NIRF) with an alkyne moiety was used to generate a 6-fluorescent glucose analog (6FGA). See FIG. 2A-D. Its ability to be transported by SGLTs selectively was then evaluated. Importantly, the speed and flexibility of click chemistry is amenable to explore various fluorophores and chelators for radionuclides and production for clinical use in the future. See FIG. 3.

3. C6-Modified Glucose Analogs are Transported by SGLTS

Click chemistry was used to create a new, C6-modified fluorescent glucose analog (6FGA) that is selectively transported by SGLTs. See FIG. 2A-D. Without wishing to be bound by theory, it serves as proof of concept that agents could be similarly radiolabeled with an imaging radionuclide such as ⁶⁸Ga and used to assess SGLT presence and function, i.e., to confirm that SGLTs are present and to quantify their ability to concentrate an agent. This would inform patient-specific treatment planning when using a therapeutic radiolabel such as ¹⁷⁷Lu. The ability of SGLTs to concentrate agents intracellularly allows low systemic concentrations and may potentially minimize toxicity. Having a mean range of 0.3 mm, the medium-energy β⁻ emissions of ¹⁷⁷Lu can penetrate many cell lengths and thereby kill adjacent cancer cells that might not express SGLTs (Emmett et al. (2017) J Med Radiat Sci. 64:52-60). This ability to effectively kill metastatic cancer cells despite tumor heterogeneity has far-reaching clinical implications. Further, this approach enables a precision medicine paradigm. By using a diagnostic label such as ⁶⁸Ga, quantitative PET imaging can provide volumetric images to confirm and quantify SGLT target presence and function. The patient-specific dosimetry provided by PET imaging would be used to provide an individualized treatment using ¹⁷⁷Lu, thereby optimizing the dose to ensure efficacy while minimizing toxicity.

4. Summary of Preliminary Work

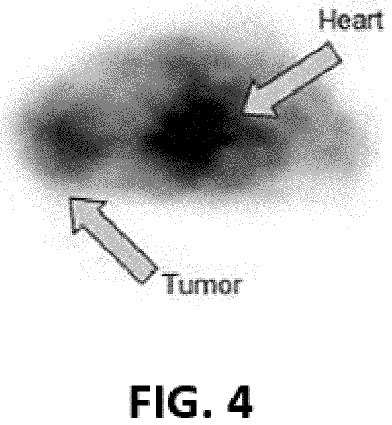

As stated above, 18F-labelled 6-fluoro-6-deoxy-D-glucose ([18F]6FDG) was previously developed as a novel imaging agent for glucose transport. This prior work focused on [18F]6FDG as a non-metabolized, in vivo glucose transport compound for use in research. Recently, it was realized its potential for imaging cancer as the relevance of SGLTs in cancer imaging and therapy is currently evolving. It was previously established that, similar to glucose, 6FDG is transported by SGLTs and GLUTs. Additionally, glucose analogs that are substrates for SGLTs are concentrated intracellularly up to 400-fold their extracellular concentration. This is more than sufficient for imaging; in routine [18F]FDG-PET scanning, three-fold concentrating (Specific Uptake Value, SUV=3 g/ml) is readily visualized. [18F]6FDG accumulation in tumor was demonstrated in a preclinical model with confirmed upregulation of mRNA for SGLT1. See FIG. 4. [18F]6FDG is reabsorbed in proximal tubules of kidneys similarly to glucose so that no activity was evident in the bladder of rats administered [18F]6FDG unless the SGLT inhibitor phlorizin was administered. However, as [18F]6FDG is transported by both SGLTs and GLUTs, its biodistribution mirrored that of glucose, and therefore lacked the SGLT specificity that is required for the proposed cancer imaging and therapy of tumor metastases.

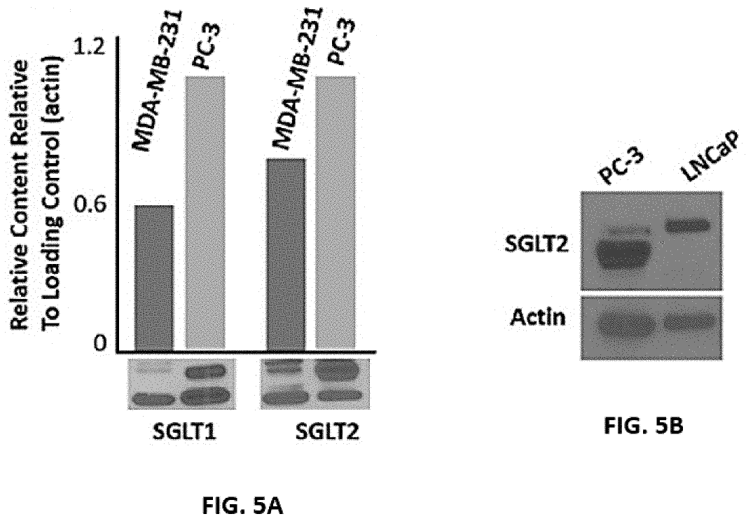

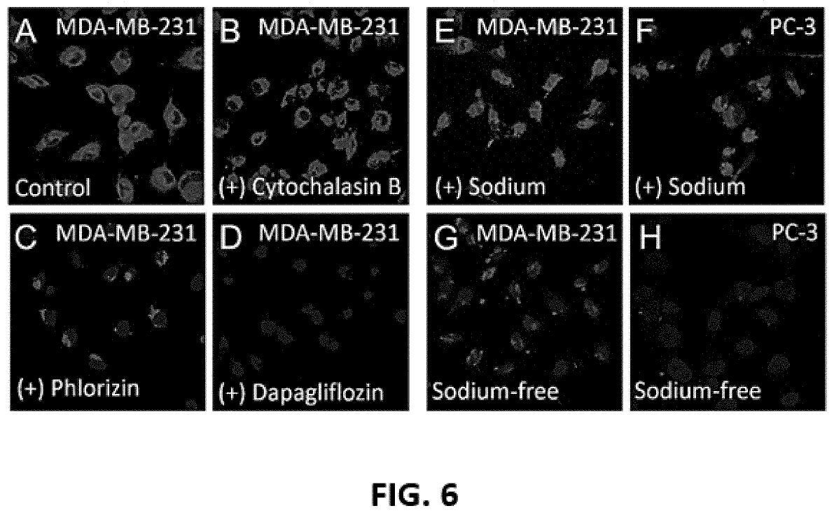

Extrapolating prior work with ¹⁸F-labelled 6-fluoro-6-deoxy-D-glucose ([¹⁸F]6FDG), it is hypothesized that another glucose analog modified on a carbon atom at position 6 might be designed to be selectively transported by SGLTs. Click chemistry was used to create a new compound, C6-modified fluorescent glucose analog (6FGA). See FIG. 2A-D. Its transport properties were evaluated in two human metastatic cancer cell lines: triple negative breast cancer MDA-MB-231 and human prostate cancer (PC-3). It is shown that: (1) both cell lines express SGLTs; (2) both concentrate 6FGA intracellularly; (3) SGLT inhibitors, including the highly SGLT2-selective dapagliflozin, block uptake; (4) GLUT inhibitors do not block uptake; and (5) presence of the co-transported molecule Na⁺ is required for uptake. Taken together, this strongly supports the new compound 6FGA is selectively transported by SGLTs and has potential as a theranostic for metastatic prostate cancer. See also FIG. 5A, FIG. 5B, and FIG. 6.

5. 6FGA Synthesis

The chemical probe was generated by click chemistry between a fluorescent dye with an alkyne moiety and azido-D-glucose. See FIG. 2A and FIG. 2B. Briefly, cyanine5.5-alkyne (1 equivalent) and 6-azido-6-deoxy-D-glucose (1 eq. were dissolved and incubated in dimethyl sulfoxide in the presence of copper sulfate (10 eq.) and THPTA (30 eq.) for 24 hours at a room temperature. The reaction was initiated with the final addition of ascorbic acid. (The target compound was isolated by reverse-phase HPLC purification using an increasing gradient of acetonitrile with 0.1% trifluoroacetic acid against water through a C-18 column. See FIG. 2C. Pooled fractions were frozen and subsequently lyophilized. The compound was characterized by mass spectrometry resulting to mass-to-ratio consistent with the expected molecular weight 826 Daltons. See FIG. 2D.

6. Selection of Prostate and Breast Cancer as Model Systems

SGLTs are overexpressed in metastatic triple-negative breast cancer (Weihua et al. (2008) Cancer cell 13:385-93) and prostate cancer (Blessing et al. (2012) J Cancer Sci Ther. 4:306-12; Scafoglio et al. (2015) Proceedings of the National Academy of Sciences of the United States of America 112:E4111-9). In FIG. 5A, Western blots using the the metastatic triple-negative breast cancer cell line MDA-MB-231 and the prostate cancer cell line PC-3 demonstrate SGLT1 and SGLT2 protein expression. FIG. 5B aso shows a second metastatic prostate cancer cell line LNCaP with preliminary westerns and antibodies against SGLT2, demonstrating expression of these transporters, consistent with previous reports.

7. SGLT-Mediated Transport of 6FGA

6FGA is transported into the SGLT-expressing cell lines for human metastatic TNBC (MDA-MB-231) and prostate cancer (PC3). In the PC3 cell line, 6FGA uptake is not blocked by the GLUT inhibitor cytochalasin B, uptake is blocked by SGLT inhibitors phlorizin and the highly SGLT2-selective dapagliflozin, and there is no uptake in the absence of Na+, the molecule that SGLTs cotransport with glucose. See FIG. 6, panels A-G. Furthermore, similar results are observed in MDA-MB-231. See FIG. 6, panels F and H. Without wishing to be bound by theory, this evidence indicates the cell uptake of 6FGA is mechanistically dependent on the presence of sodium, and thus transported by SGLTs, and potentially avoids off-target effects of non-selective glucose analogs also transported by GLUTs.

8. 6FGA does not Cross the Blood-Brain Barrier

A potential concern for a therapeutic glucose analog radionuclide is the agent might concentrate in the brain or other normal tissues and cause treatment related CNS or other toxicities. FIG. 7 demonstrates distribution of 6FGA at 24 hours post-injection, localizing mostly in the tumor with low background in the kidneys and intestines where SGLT transporters are found physiologically. Importantly, 6FGA was not found in the brain. An intact blood-brain barrier, which lacks SGLTs should not transport SGLT-selective glucose analogs.

9. 6FGA is Retained in Tumors

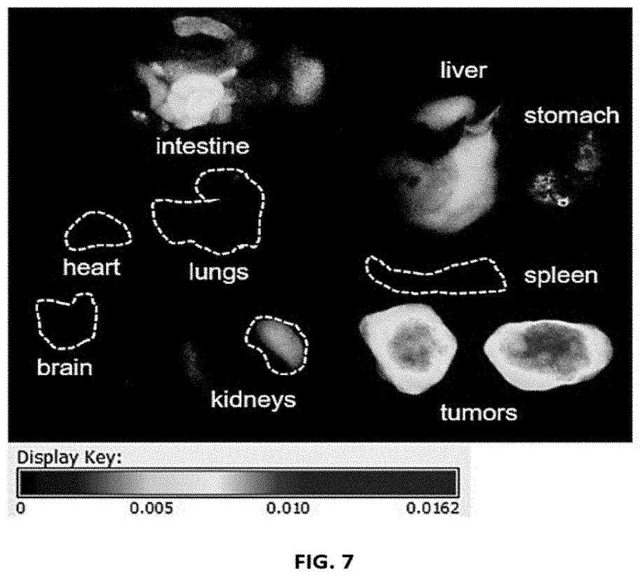

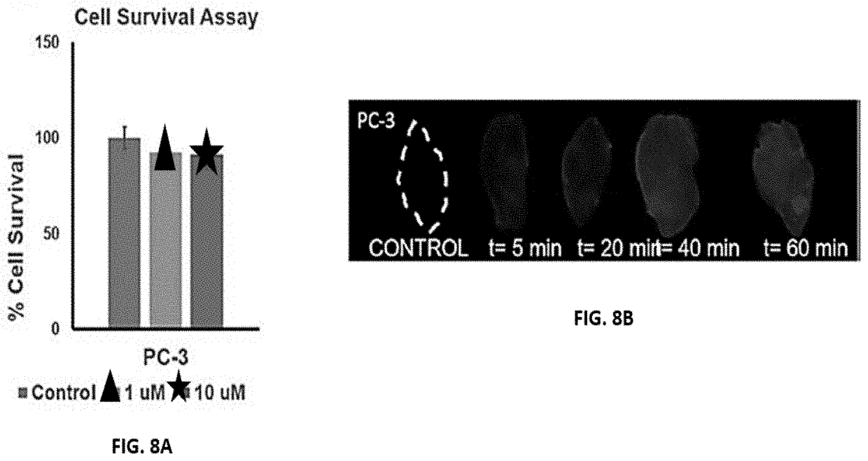

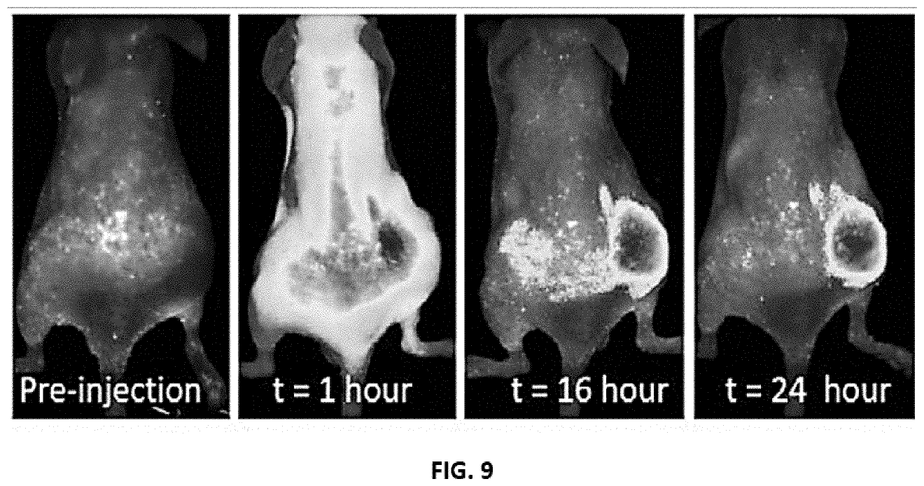

In ex vivo imaging experiments, the 6FGA signal is retained, without washout, over the 60-minute observation interval. See FIG. 8A and FIG. 8B. Finally, 6FGA concentrates in vivo and is visualized in a murine model of human prostate cancer, with very little distribution in normal tissues at 16 hrs and 24 hrs respectively. See FIG. 9. Without wishing to be bound by theory, these evidence suggest that 6FGA is selectively transported by SGLTs and demonstrates biologic properties that support translation to in vivo cancer imaging and therapy.

10. Synthesis of Precursor for Radiolabeled Glucose Analogue Targeting Sodium Dependent Glucose Transporter

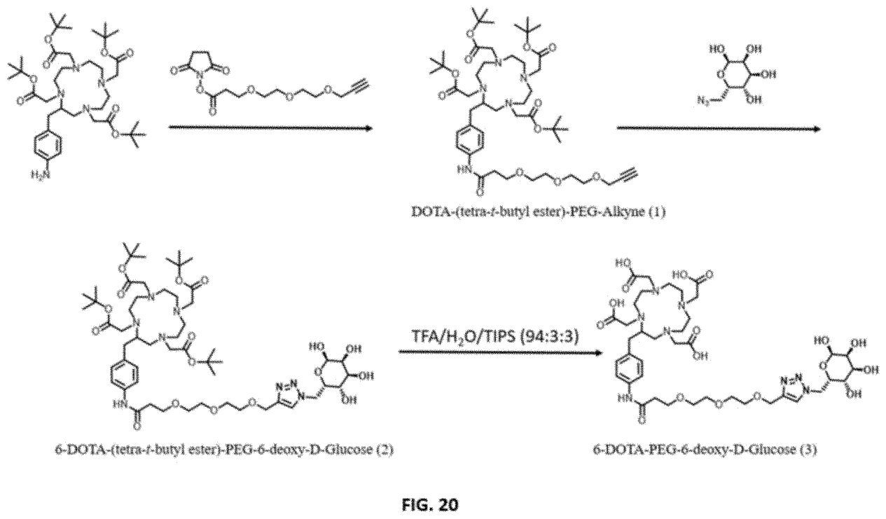

With reference to FIG. 20, a fully protected radiolabeled DOTA ligand was coupled to a pegylated alkyne by amide formation to generate a “clickable” agent (1) with a linker that aids in solubility in aqueous solutions. The DOTA analogue (1) was conjugated to 6-azido-deoxy-D-glucose via a copper-assisted Huisgen cycloaddition to generate 2. The DOTA moiety of 2 deprotected by removal of the tert-butyl esters in the presence of trifluoroacetic acid, generating the radiolabeling precursor glucose analogue 3.

Para-amino-benzyl-DOTA-tetra(tButyl ester) (cat #B-201, Macrocyclics, Dallas, Tex.) was dissolved in dimethyl sulfoxide with 3 equivalents of N,N-diisopropylethylamine and 3 equivalents of propargyl-PEG3-NHS ester (cat # BP-21613, BroadPharm) and stirred overnight at 50° C. The reaction mixture was coarsely purified with C18 solid phase extraction column that was primed with acetonitrile followed by water. Impurities were eluted with water and 20% acetonitrile in water. The coupling reaction product was finally eluted with 50% acetonitrile and further purified with reversed-phase HPLC (Luna 5 micron C18-250×10 mm column) with 0.1% trifluoroacetic acid in water and 0.1% trifluoroacetic acid in acetonitrile as the mobile phase. The major peak detected at 254 and 280 nm was collected and the pooled fractions were lyophilized. ESI-MS, calculated from C49H81N5O12 m/z 931.59; found [M+Na] 954.59.

Copper-assisted azide-alkyne Huisgen cycloaddition was employed to generate 2. Three-fold of excess 6-azido-6-deoxy-D-glucose (Biosynth Carbosynth, UK) was dissolved with 1 in dimethyl sulfoxide. Catalytic amount of THPTA-copper complex was added with 20 equivalents of copper sulfate. The reaction was finally initiated upon addition of ascorbic acid. After a 24-hour incubation at room temperature, the crude mixture was coarsely purified with C18 solid phase extraction upon which excess ascorbic acid and copper sulfate were eluted with 25% acetonitrile in water. The product was eluted from the C18 plug with 50% acetonitrile in water and further purified with reversed-phase HPLC (Luna 5 micron C18-250×10 mm column) with 0.1% trifluoroacetic acid in water and 0.1% trifluoroacetic acid in acetonitrile as the mobile phase. The major peak detected at 254 and 280 nm was collected and the pooled fractions were lyophilized. ESI-MS, calculated from C55H92N8O17 m/z 1136.66; found [M+Na] 1159.

For the removal of the t-butyl esters, 2 was mixed in a solution of 94% trifluoroacetic acid, 3% water, and 3% triisopropylsilane. After mixing for 24 hours, the trifluoroacetic acid was evaporated under a gentle flow of air. The product was isolated by reversed-phase HPLC (Luna 5 micron C18-250×10 mm column) with 0.1% trifluoroacetic acid in water and 0.1% trifluoroacetic acid in acetonitrile as the mobile phase. The major peaks detected at 254 and 280 nm was collected and the pooled fractions were lyophilized. ESI-MS, calculated from C39H60N8O17 m/z 912.41; found [M+H] 913 and [M+Na] 935.

### G. Prophetic Examples

1. Quantification of SGLT-Mediated Uptake of 6FGA by Human Cancer Cells In Vitro.

SGLTs, expressed in numerous cancers, can concentrate glucose intracellularly to many fold its external concentration and, thus, are promising for delivering imaging and therapeutic agents. Preliminary work using two cell lines provides strong evidence evidence of SGLT-mediated uptake specifically in metastatic TNBC (MDA-MB-231) and prostate cancer (PC-3). The evaluation will be extended to other breast and prostate cancer lines, use of shRNA knockdown of SGLT, selective and non-selective SGLT and GLUT inhibitors, and transport assays in the absence of Na, to unambiguously verify that this uptake is is due to SGLTs, and identify the most appropriate cell lines for in vivo evaluation.

a. SGLT Expression in Cancer Cells

Studies are carried out using metastatic cancer cells from validated TNBC and prostate cancer panels obtained from American Tissue Culture Collection (ATCC). Expression levels of SGLT1 and SGLT2 and GLUT1 of each cell line are determined by RT-PCR and immunoblots against the corresponding antibodies in comparison to housekeeping protein (e.g., actin). As controls, the expression levels of SGLTs are compared to high SGLT expressing normal liver cells and low SGLT expressing normal kidney cells (Zaidi et al. (2012) Virus genes 44:1-7). From these selections, high and low expressing SGLT cell lines are examined for function using transport assays.

b. SGLT-Mediated Transport Assays

The assays are performed from a modified protocol as we have done previously, and from flow cytometry formats (Zou ry al. (2005) J Biochem Biophys Methods 64:207-15; Landau et al. (2007) American journal of physiology Endocrinology and metabolism 293:E237-45). In brief, cells are grown to confluence using recommended growth media in 96-well plates. The cells are washed with PBS and incubated with 2.5 μM of 6FGA for 20 minutes at 37° C. in humidified incubator in the presence or absence of inhibitors for SGLTs (e.g., phlorizin for SGLT1 & 2, dapagliflozin which is highly selective for SGLT2) and GLUTs (e.g., cytochalasin B for GLUT1, 2, 3, and 4). The cells are detached with trypsin and resuspended in PBS. The cells are analysed using flow cytometry with the Cy5.5 filter set (excitation 685 nm, exmission 710 nm) gated for 250,000 counts. 6FGA uptake that is inhibited by phlorizin and dapagliflozin and by the absence of Na+ from the media is interpreted as being mediated by SGLTs whereas cytochalasin B-inhibitable uptake is attributed to GLUTs. FIG. 6 shows an example of such results. Uptake assays are also analysed by confocal fluorescence microscopy with cells grown on coverslips and exposed to 6FGA and inhibitors as described above.

In addition, to assess the potential for acidic tumor microenvironment, 6FGA uptake is assessed in normal and low pH conditions. Transport assays are performed at pH 5.0, 6.0, 7.0, and 7.4. In order to assess the microscopic distribution of 6FGA, cells grown on coverslips and subject to the various inhibition conditions, described above, will be imaged using confocal fluorescence microscopy.

C. Western Blot Analysis

SGLTs and GLUTs content are obtained from cell lysates. Standard gel electrophoretic separation and transfer to membranes are performed. Membranes are probed for SGLT1, SGLT2, and GLUT1 using the corresponding primary and secondary antibodies and are visualized using ECL. Immunoblots are normalized against actin.

d. RT-PCR

The expression of SGLTs and GLUTs are measured using RT-PCR. Total RNA are extracted from cultured cells using the RNeasy kit (Qiagen). Total mRNA are reverse transcribed to cDNA (High Capacity cDNA Reverse Transcription Kit, Applied Biosystems). The cDNAs are amplified using primers for the target genes (SGLT1, SGLT2, GLUT1, and GLUT4) and GAPDH as internal control using optimized conditions (Helmke et al. (2004) Oral oncology 40:28-35).

e. Shrna Experiments

To confirm that the uptake is mediated by SGLTs, uptake assays are also be performed in cells with SGLT1 and SGLT2 knock down using shRNA transfection (Blessing et al. (2012) J Cancer Sci Ther. 4:306-12). Vectors expressing shRNA target sequence or the corresponding scrambled sequences are be transfected by standard techniques. The knockdown is verified by western blot analyses and immunocytochemical analysis.

f. ANALYSES

Immunoblots are processed using Image J (NIH). Experiments are performed in triplicates and repeated at least twice. Data are presented with mean and SD and the significance determined by t-test.

2. Evaluation of the In Vivo Biodistribution of 6FGA Via Imaging Preclinical Models of Human Cancer

Preclinical models are the natural transition between studies in cells and humans. Further, tissues can be collected and analyzed using IHC and RT-PCR to corroborate the mechanism of uptake observed by fluorescent imaging and cell uptake assays as detailed above.

The purpose is to show that SGLT is targetable in vivo. For consistency, the same modifications of the parental cell line are used across all prophetic examples. Namely, cancer cells identified above are are transfected with imaging reporter gene expressing vectors (green fluorescent protein and luciferase). Mice are inoculated with the cells to create subcutaneous flank tumors. Once tumors reach appropriate size as determined via caliper measurements, fluorescence imaging is used to assess the in vivo concentration of 6FGA and demonstrate localization in SGLT-expressing tumors, visually and quantitatively. Tumors are harvested, expression levels of SGLTs and GLUTs are confirmed using whole tissue lysates for western blots, immunohistochemistry and qRT-PCR. Evidence for SGLTs as the mechanism of uptake is determined by (1) decreased uptake in the presence of pharmacologic and shRNA SGLT inhibition and (2) equal uptake with GLUT inhibition.

For practicality, experiments are conducted using subcutaneous tumors, both without and with (±) pharmacologic inhibitors and shRNA knockdown. In preparation, a small cohort of six tumor-bearing mice are imaged to determine the tumor uptake time for optimal 6FGA signal.

For the pharmacologic inhibition studies, each mouse is evaluated for 6FGA uptake in the absence and presence (randomized order) of pharmacologic inhibitor so that each animal serves as its own control: groups 1 through 3 in Table 1. For example, in group 1 each mouse is scanned in two imaging sessions, once with and once without (+) the SGLT inhibitor phlorizin. The order of the with (+) and without (−) inhibitor studies is randomized, allowing time for 6FGA clearance between studies, for anesthetic recovery and restitution from effects of inhibitor, but otherwise minimizing the potential for changes in tumors. Other groups are used for the other two inhibitors, dapagliflozin (highly selective SGLT2) and cytochalasin B (GLUT). shRNA inhibition studies are also conducted to further confirm the SGLT-driven mechanism of uptake. There are groups with shRNA knockdown of SGLT1, SGLT2, and both SGLT1 and SGLT2 in the parental cell line, as summarized in Table 1. For these the animals without knockdown and without (−) inhibitor, groups 1 to 3, serve as controls.

TABLE 1

| Exper. | 1 | 2 | 3 | 4 | 5 | 6 |
|---|---|---|---|---|---|---|
| shRNA | − | − | − | 1 | 2 | 1&2 |
| Phlor | ± | − | − | − | − | − |
| Dapa | − | ± | − | − | − | − |
| Cyto B | − | − | ± | − | − | − |

shRNA = SGLT knockdown, Phlor = SGLT inhibitor phlorizin, Dapa = SGLT2 inhibitor dapagliflozin, Cyto B = GLUT inhibitor cytochalasin B.

To validate the in vivo imaging results, a separate study is conducted wherein, immediately after imaging, mice are euthanized and tissues harvested, and subject to ex vivo imaging and fluorimetry. To span the range of concentrations expected, mice are imaged at 1 h and at 24 h after 6FGA administration. Groups are shown in Table 2.

TABLE 2

| Group | 1 | 2 | 3 | 4 | 5 | 6 | 7 | 8 | 9 | 10 | 11 | 12 |
|---|---|---|---|---|---|---|---|---|---|---|---|---|
| Cancer | B | B | B | B | B | B | P | P | P | P | P | P |
| shRNA | − | − | − | + | + | + | − | − | − | + | + | + |
| Phlor | ± | | | ± | | | ± | | | ± | | |
| Dapa | | ± | | | ± | | | ± | | | ± | |
| Cyto B | | | ± | | | ± | | | ± | | | ± |

B = triple-negative breast cancer, P = prostate cancer; shRNA = SGLT knockdown, Phlor = SGLT inhibitor phlorizin, Dapa = SGLT2 inhibitor dapagliflozin, Cyto B = GLUT inhibitor cytochalasin B.

a. Cancer Models

A selected prostate cancer cell line as disclosed above is transfected with imaging reporter gene expressing vectors (green fluorescent protein and luciferase).

Subcutaneous flank tumors are grown in athymic NCr nu/nu immunosuppressed mice. Tumor growth is monitored by visual inspection and caliper measurement. When the volume reaches appropriate size, imaging studies are conducted.

b. In Vivo Quantitative Fluorescence Imaging

Using methods previously employed, mice are placed on an alpha-free diet for 7 to 10 days prior to imaging to reduce autofluorescence signal. After 6FGA injection, the mice are imaged using a Maestro In Vivo Imaging System (CRi Inc, Woburn, Mass.). With the corresponding filter sets, multispectral images are acquired and a spectral library is generated by assigning 6FGA fluorescence from autofluorescence. After quantitative unmixing of multispectral image, regions of interests (flank tumor) are defined for comparisons of intensity among different treatments.

c. Ex Vivo Fluorescence Imaging

Applying techniques previously employed, ex vivo imaging is performed following in vivo imaging to corroborate image-derived measures of 6FGA uptake. To span the range of concentrations expected, the validations will be done under the conditions indicated in Table 2. Immediately after imaging, mice are euthanized, tumors collected, and sectioned for ex vivo imaging, e.g., FIG. 7. Cryopreserved tumor tissues are sectioned for immunofluorescence staining for SGLT and analyzed with fluorescence confocal microscopy for the uptake of 6FGA in tumor cells.

d. Fluorimetry

Briefly, tumors samples are homogenized in acidified ethanolic solution and centrifuged at 5,000 rpm for 10 min at 4° C. Aliquots of the supernatant are placed in black, 96-well plates and read in fluorescence microplate reader (excitation 685 nm, emission 710 nm). 6FGA concentration in tumor is determined by a standard curve made of 6FGA and standardized by sample weight.

e. Immunofluorescence

Frozen sections are prepared from fresh tissues that are fixed in 4% paraformaldehyde and cryopreserved in 30% sucrose before freezing embedded in Optimal Cutting Temperature (OCT) compound. The tissues are sectioned to 10-20 microns in a cryostat on slides and warmed to room temperature. Primary antibodies for SGLT1 and SGLT2 are applied for 90 min (at required dilution). fluorochrome conjugated secondary antibody is incubated for 30 min at room temperature. Nuclei is counterstained with 4′, 6-diamidino-2-phenylindole (DAPI). Processed tissue sections are imaged with confocal fluorescence microscopy for SGLTs, nuclei, and 6FGA.

f. Data Analysis

Regions of interest (ROIs) are manually delineated by investigators experienced with preclinical imaging studies. ROI mean, standard deviation, and other summary statistics are calculated from pixel values calibrated to % ID as described above. A statistically significant reduction of 6FGA uptake in presence of SGLT inhibitor or knockdown supports that SGLT drives 6FGA uptake.

g. Numbers of Mice and Statistical Significance

Differences in % ID and other parameters are evaluated for significance.

Measurements from the same mice scanned in two conditions, e.g., control vs. with inhibitor, are compared using a Wilcoxon Signed-Rank test. Measurements in different mice, e.g., without and with shRNA knockdown, are compared using a Mann-Whitney U test. A one-sided, α=0.05 test is used to declare statistical significance. The sample size of 10 mice per group achieves 69% or greater power to detect a factor-of-two difference in uptake between any two groups. Adjustment for multiple testing using the false discovery rate approach is used. Table 4 shows 20 groups of 10 plus an additional 6 mice to determine the optimal time for imaging.

3. Evaluation of the In Vivo Biodistribution of 6FGA in Metastases and Normal Tissues

Without wishing to be bound by theory, the goal is to demonstrate SGLT-expressing metastases concentrate and retain 6FGA whereas normal tissues have low concentration, maximizing treatment efficacy and minimizing off-target toxicity. Here, three techniques will be utilized: (1) perform whole-animal, dynamic, fluorescence molecular tomography (FMT) imaging to monitor systemic clearance in a subcutaneous flank tumor model of human prostate cancer. This determines optimal time points needed to accurately quantify 6FGA kinetics; (2) use ex vivo fluorescence imaging and fluorimetry of tissues from ssignedraft model euthanized at specified time points to quantify 6FGA concentration time course; and (3) use whole-mouse, three-dimensional (3D) cryo-imaging of a metastatic model to evaluate 6FGA distribution in prostate cancer metastases and normal tissues. To evaluate the potential for therapeutic efficacy and future clinical translation, the biodistribution data will be treated as that of a hypothetical ¹⁷⁷Lu radiolabeled analog and radiation dose and expected cell kill are calculated. FIG. 7 and FIG. 9-11 support feasibility of the methods described below.

In (1) and (2), subcutaneous flank tumor models are created as detailed above. In (3), models of human metastatic prostate cancer are generated as detailed below. All experiments use cell lines modified with reporter gene expressing vectors (green fluorescent protein and luciferase). In (3), bioluminescence is used to monitor in vivo metastatic tumor progression to identify the appropriate time for the 6FGA biodistribution studies. In (1), semi-quantitative, dynamic FMT imaging is performed to collect 6FGA time course until the fluorescent signal becomes negligible, out to at least 2 days based results shown in FIG. 9. A dense sampling schedule is used in order to determine the number and scale of exponentials to model in the retention curves. These are used to determine a practically-optimized sampling schedule for the other subaims. For example, a biexponential necessitates 4 to 6 samples. (2) is the follow-up experiment in which mice are euthanized at specified time points, tissue sections quantitatively imaged, then homogenized for fluorimetric analysis. Images provide spatial distribution within tumor and normal tissues. Fluorimetry results are calibrated to absolute concentration then normalized by injected dose. Percent injected dose (% ID) versus time data for each tissue is modeled as sum of the minimum required number of exponentials, and integrated to determine area under the curve (AUC). Using the Medical Internal Radiation Dose (MIRD) formalism, organ radiation dose is predicted assuming that 6FGA represents a hypothetical, ¹⁷⁷Lu-labeled therapeutic. These are analogous to previous work evaluating in vivo biodistribution in rodents and extrapolation to humans.

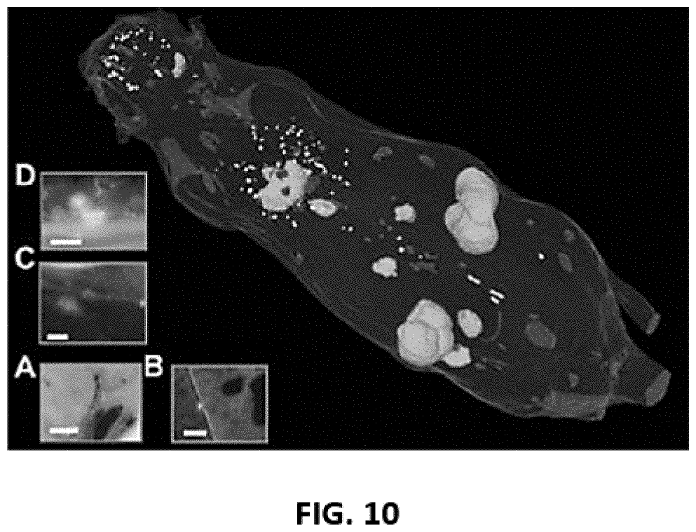

For (3), a metastatic model is used and the metastatic sites are expected to be small and appear at numerous locations throughout the body. Thus, whole-mouse cryo-imaging is used where cancer cells expressing green fluorescent protein (GFP) and the near infrared (NIR) fluorescence of 6FGA are imaged, e.g., FIG. 10. Concordance of GFP and 6FGA signals is analyzed to predict the therapeutic efficacy taking into account the beta particle range of ¹⁷⁷Lu should provide a targeted radionuclide therapy that is efficacious even with heterogeneous cancer. Details are given below.

More specifically, in (1), a group of mice with prostate cancer in subcutaneous flank tumors is used. Using FMT, mice are imaged longitudinally, over multiple days, to determine the optimal sampling times needed in B. In (2), groups of mice are euthanized at each of the specified sampling times. A priori, we anticipate six time points are needed. Ten mice are prepared for each group to hedge against mortality, to sample the variability in tumors, and to achieve a minimum of five good measurements per time point. Harvested tissues are subject to ex vivo imaging and fluorimetry. In (3), two groups of mice are used corresponding to an early and late time point. The early time point is selected to provide visualization of normal tissue biodistribution and the late point for retention in tumor.

a. Cancer Models

Prostate cancer cells are selected in the experiments described above and transfected with reporter gene expressing vectors (GFP, luciferase) (also as described above). For metastatic models, cancer cells are introduced into athymic NCr nu/nu or SCID mice through the tail vein, prostatic, or left ventricle injection as appropriate for the selected cell line. Tumor growth is identified and monitored by bioluminescence imaging following luciferin injection.

b. Fluorescence Molecular Tomography (FMT) Imaging

Mice are placed on an alpha-free diet for 7 to 10 days prior to imaging to reduce the autofluorescence signal. They are placed on the cassette and imaged using a FMT 2500 scanner (Perkin-Elmer, Waltham, Mass.). 6FGA fluorescence is calibrated for the instrument to support determination of % ID within the tumor volume and other selected tissues using methods that the team has previously described.

c. Ex Vivo 6FGA Imaging and Fluorimetry

Groups of mice are euthanized at each selected time point by cervical dislocation under anesthesia and tumor and normal tissues immediately harvested and imaged using a Maestro In Vivo Imaging System (CRi Inc, Woburn, Mass.) to produce spatial concentration maps of 6FGA within each tissue as detailed in above. Subsequently, tissues are homogenized and total tissue concentration determined by fluorimetry, analogous to that of tumors, as described above.

d. Data Analysis

To evaluate the clinical feasibility of SGLT-targeted radionuclide therapy using a hypothetical ¹⁷⁷Lu labeled glucose analog, time-concentration curves generated from fluorimetry data are extrapolated to predict normal tissue radiation doses for humans. In brief, the curves are normalized for injected dose and sample size, and extrapolated to that for humans. Exponential models are fit to the resultant curves and areas under the curves (AUCs) estimated. AUCs correspond to residence times from which absorbed dose (energy deposition per unit mass) is calculated using the Medical Internal Radiation Dose (MIRD) formalism. It is implemented in, e.g., OLINDA/EXM (see https.//pubmed.ncbi.nlm.nih.gov/15937315/), which accounts for tissue self-dose as well as dose to other tissues based on S-factors and assumed nominal patient geometry.

e. Whole-Mouse Ex Vivo Cryo-Imaging

When appropriate metastatic burden is reached as determined using bioluminescence imaging, 6FGA is injected and groups of mice are subsequently euthanized at early and late time points. 3D cryo-imaging (CryoViz, BiolnVision, Inc., Cleveland, Ohio) is then used to enable high resolution, high sensitivity imaging of 6FGA needed to identify metastases at unknown locations. In brief, to prepare for cryo-imaging and to prevent trapped air bubbles, fresh mice carcasses are rubbed with Optimal Cutting Temperature (OCT, Tissue-Tek, Terrance, CA) compound, immersed in it, and then frozen using liquid nitrogen. Cryo-imaging then uses a section-and-image technique to obtain ana-tomical color and molecular fluorescence, single cell sensitivity, 3D microscopic imaging over volumes as large as a mouse. An example is shown in FIG. 10.

f. Data Analysis

Co-registered cryo-image volumes are analyzed for the ability of SGLTs to be used to target prostate cancer metastases. One volume shows GFP-labeled tumor and another shows 6FGA in the NIR range. To quantitatively assess tumors successfully targeted by 6FGA, a 3D Rose SNR (SNR-R) is used. Using a threshold of SNR-R>4, tumors are deemed as being targeted. A histogram of the percentage of metastases successfully targeted as a function of tumor size provides summary visualization of the anticipated large number of metastatic sites.

g. Microscopic Radiation Dose and Therapeutic Efficacy

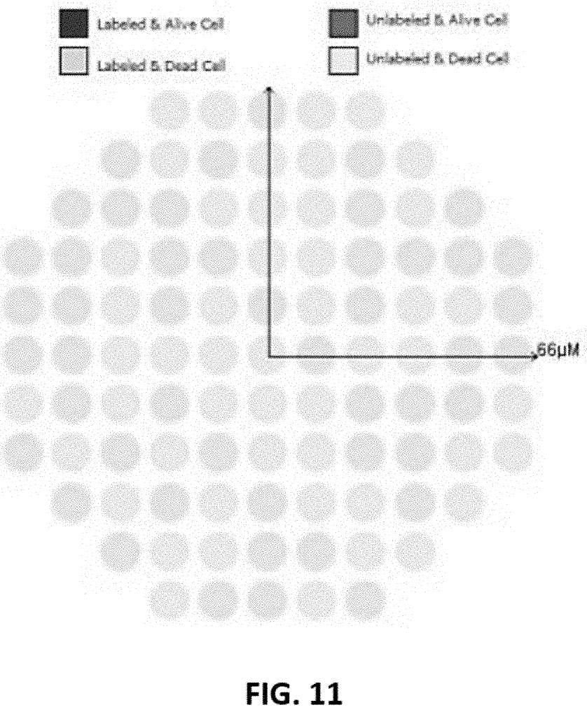

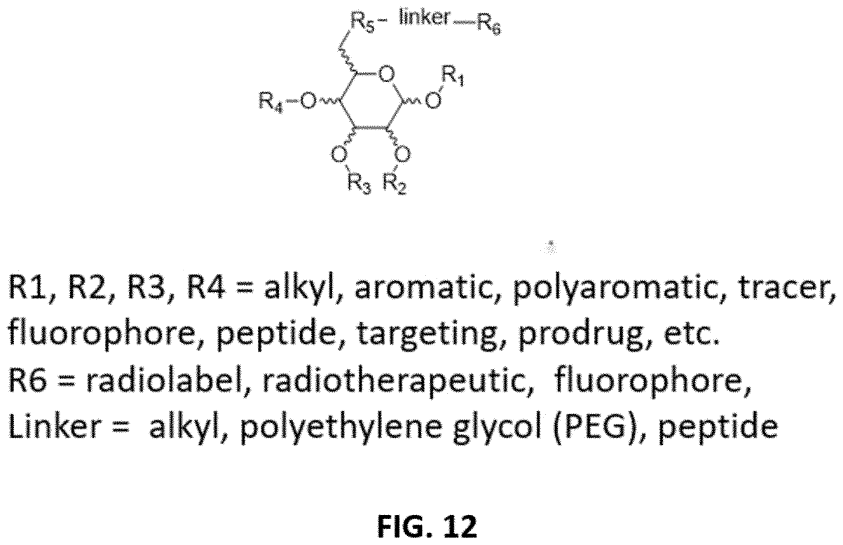

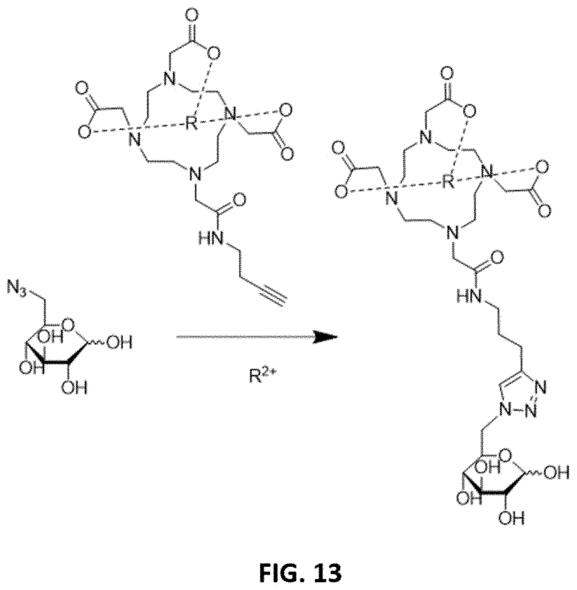

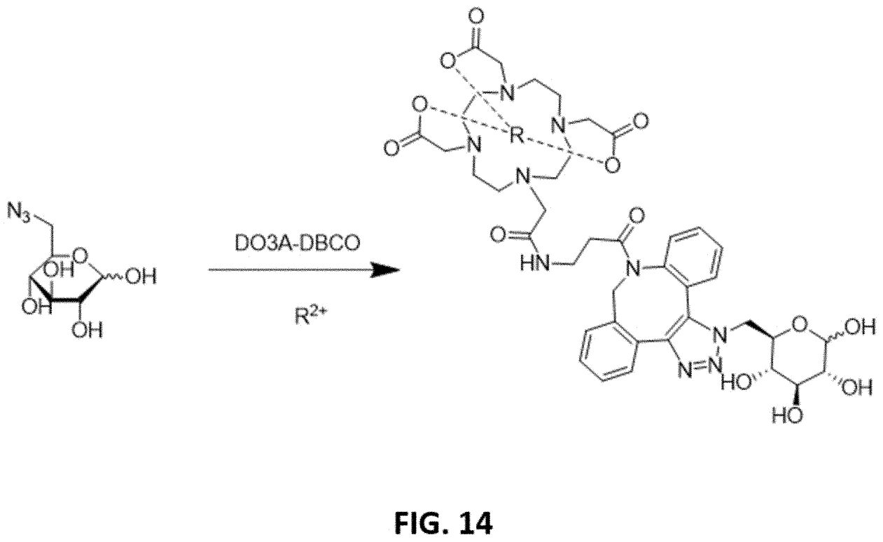

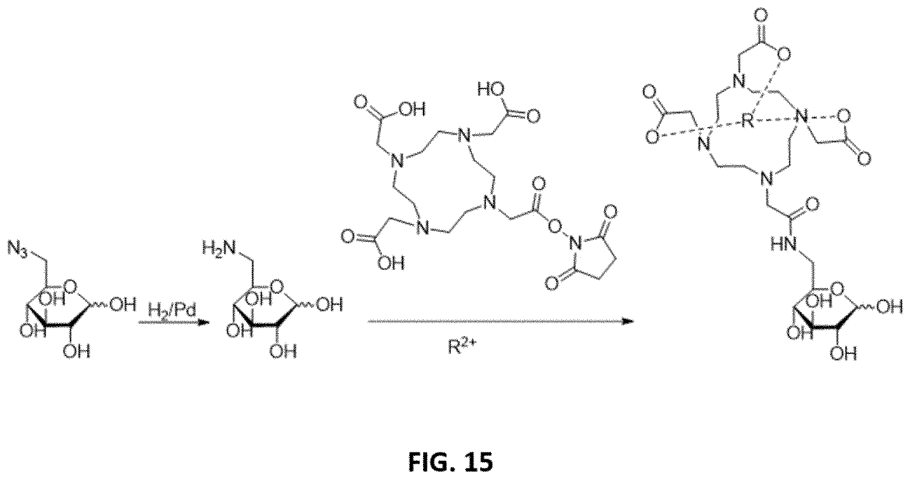

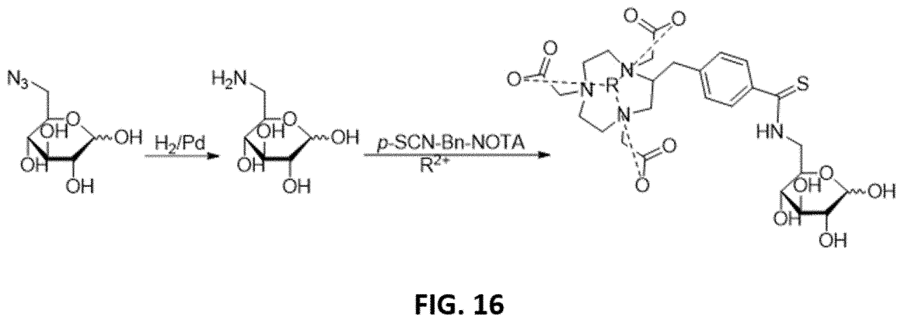

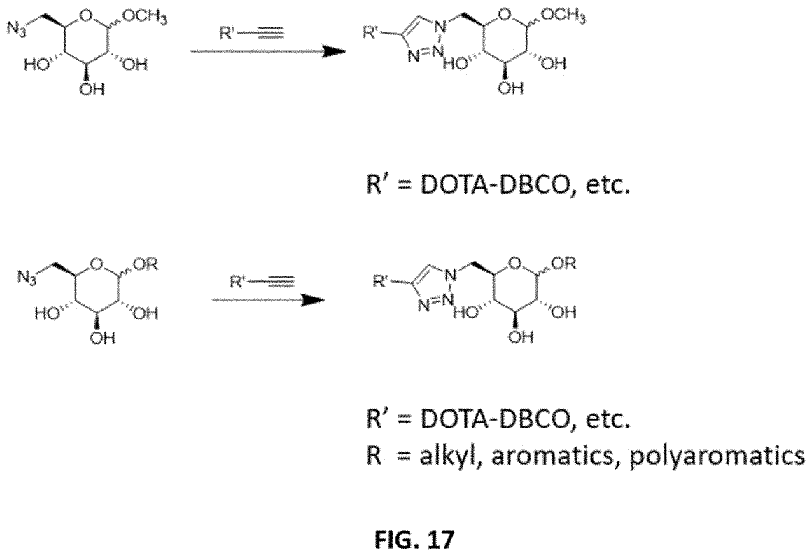

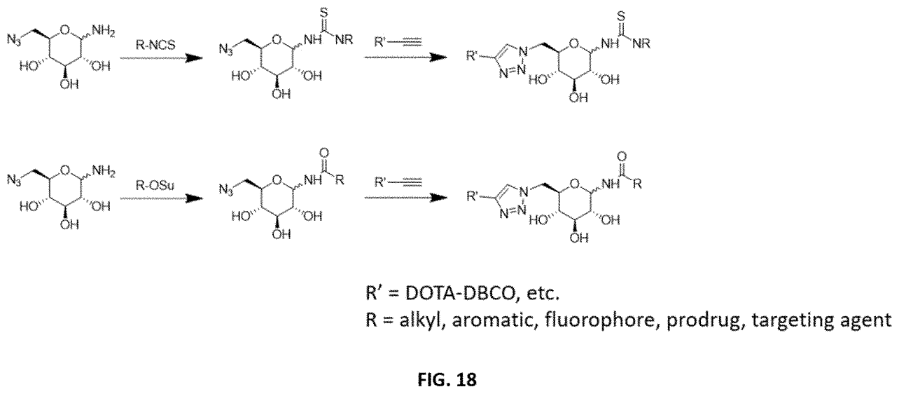

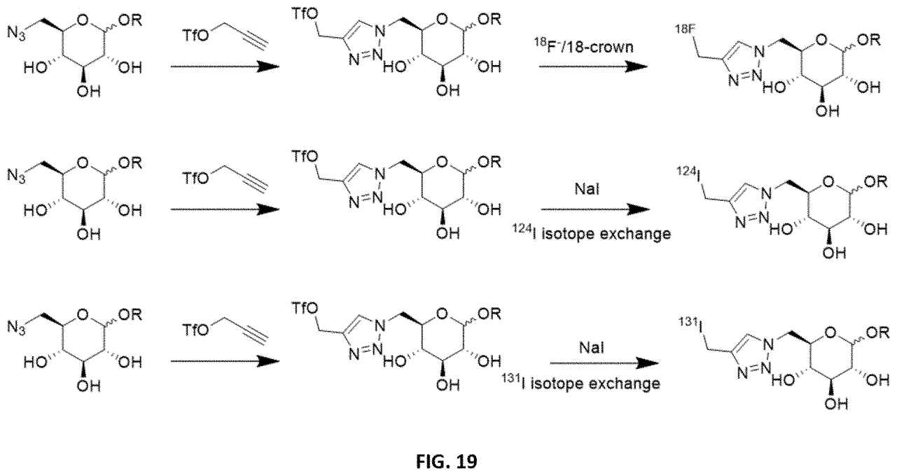

Additional analyses are used to predict therapeutic efficacy of SGLT targeting as DNA damage and cell kill extends multiple cell lengths beyond cells that express SGLTs and take up the agent. For example, beta particles emitted from a 6FGA analog therapeutically-labeled with ¹⁷⁷Lu have a mean range of 300 microns. Thus, microscopic radiation dose and cell-kill are evaluated using MIRDcell (see https://pubmed.ncbi.nlm.nih.gov/25012457/). This calculation predicts if cells will survive or die in response to local energy deposition. This uses the linear-quadratic model parameterized by a and p to predict tumor control and normal tissue complications. Values are available from the literature, but, if needed, could be determined in the laboratory. MIRDcell models a variety of cell geometric arrangements and the fraction which are radiolabeled in order to predict which cells are alive and dead. See FIG. 11. In this way, it can account for the genetic heterogeneity that manifests itself by not all of the cells containing the SGLT target.

h. Numbers of Mice and Statistical Significance

For (1), to determine the optimal time points, 10 mice will be inoculated to grow xenografts. This number is not driven to achieve a specific statistical power. It is selected to hedge against mortality, to sample the variability in tumors, and to achieve a minimum of 5 good dynamic time course image sets (60). For (2), to quantify 6FGA concentration time course, the initial group size is analogously 10 mice for each of the anticipated 6 time points. For (3), whole mouse cryo-imaging of metastatic cancer, 10 mice are imaged at early and late time-points. It is likely that more than 100 tumors per mouse will be obtained, giving a large number of “tumor experiments” to determine targeting efficiency. Assuming a nominal targeting of 0.90 for tumors >0.5 mm and 400 tumors, the confidence interval will be 0.86 to 0.92, allowing conclusions about targeting of 6FGA in tumors to be drawn by cryo-imaging.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A compound having a structure represented by a formula: 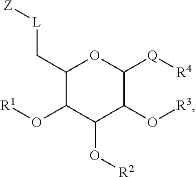
wherein L is a divalent linker;
wherein Q is selected from —O—, —NHC(O)—, and —NHC(S)NH—;
wherein Z is a fluorophore, a radiolabel, a radioisotope, or a radiotherapeutic; and
wherein each of R¹, R², R³, and R⁴ is independently selected from hydrogen, C1-C4 alkyl, aryl, a fluorophore residue, a peptide residue, and a polyaromatic residue,
provided that when Q is —O— and Z is a fluorophore, then at least one of R¹, R², R³, and R⁴ is not hydrogen,
or a pharmaceutically acceptable salt thereof.

2. The compound of claim 1, wherein Q is —O—, Z is a fluorophore, and at least two of R¹, R², R³, and R⁴ are not hydrogen.

3. The compound of claim 1, wherein each of R¹, R², R³, and R⁴ is hydrogen.

4. The compound of claim 1, wherein Z is a radiolabel, a radioisotope, or a radiotherapeutic.

5. The compound of claim 1, wherein Z is a radiolabel.

6. The compound of claim 5, wherein the radiolabel comprises a ligand and a metal.

7. The compound of claim 6, wherein the ligand is selected from DOTA, NOTA, DFO, and DOTA-DBCO.

8. The compound of claim 6, wherein the metal is selected from ⁶⁸Ga, ⁶⁴Cu, ¹⁷⁷Lu, ⁸⁹Zr, ⁸⁶Y, ⁹⁰Y, ⁴³Sc, ⁴⁴Sc, ¹⁵²Tb, ⁸²Rb, ²²⁵Ac, ²¹¹At, ²²⁷Th, ²¹³Bi, ²²⁴Ra, ²²³Ra, ⁸⁹Sr, and ¹⁵³Sm.

9. The compound of claim 1, wherein Z is a radioisotope selected from ¹⁸F, ¹²⁴I, and ¹³¹I.

10. The compound of claim 1, wherein Z is a fluorophore having a structure represented by a formula selected from:

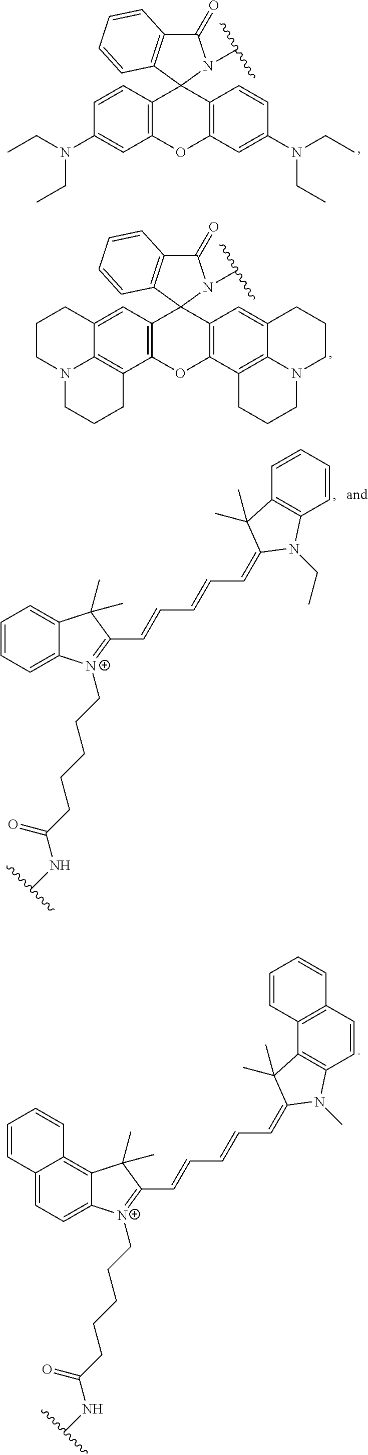 [SMILES: *N1C(=O)c2ccccc2C12c1ccc(N(CC)CC)cc1Oc1cc(N(CC)CC)ccc12 ; *N1C(=O)c2ccccc2C12c1cc3c4c(c1Oc1c2cc2c5c1CCCN5CCC2)CCCN4CCC3 ; *NC(=O)CCCCC[N+]1=C(/C=C/C=C/C=C2/N(CC)c3ccccc3C2(C)C)C(C)(C)c2ccccc21 ; *NC(=O)CCCCC[N+]1=C(/C=C/C=C/C=C2/N(C)c3ccc4ccccc4c3C2(C)C)C(C)(C)c2c1ccc1ccccc21]

11. The compound of claim 1, wherein the compound has a structure selected from:

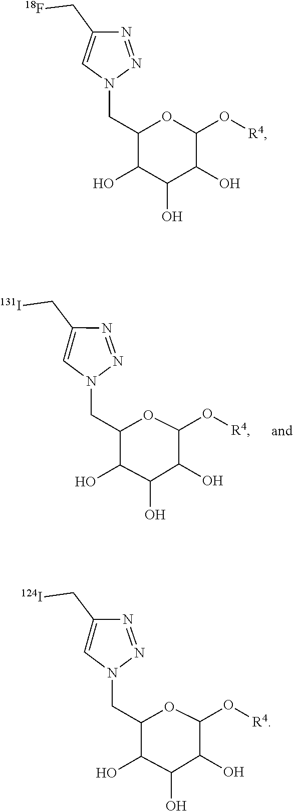

12. The compound of claim 1, wherein the compound has a structure selected from:

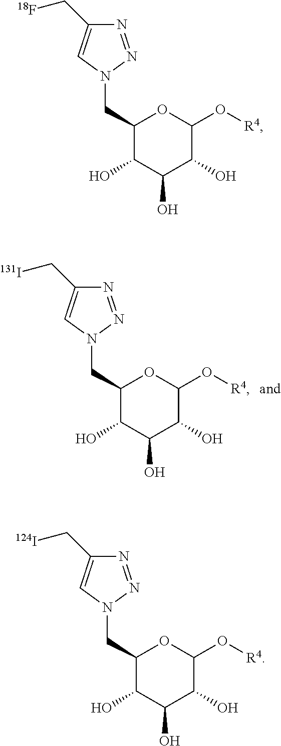

13. The compound of claim 1, wherein the compound has a structure selected from: 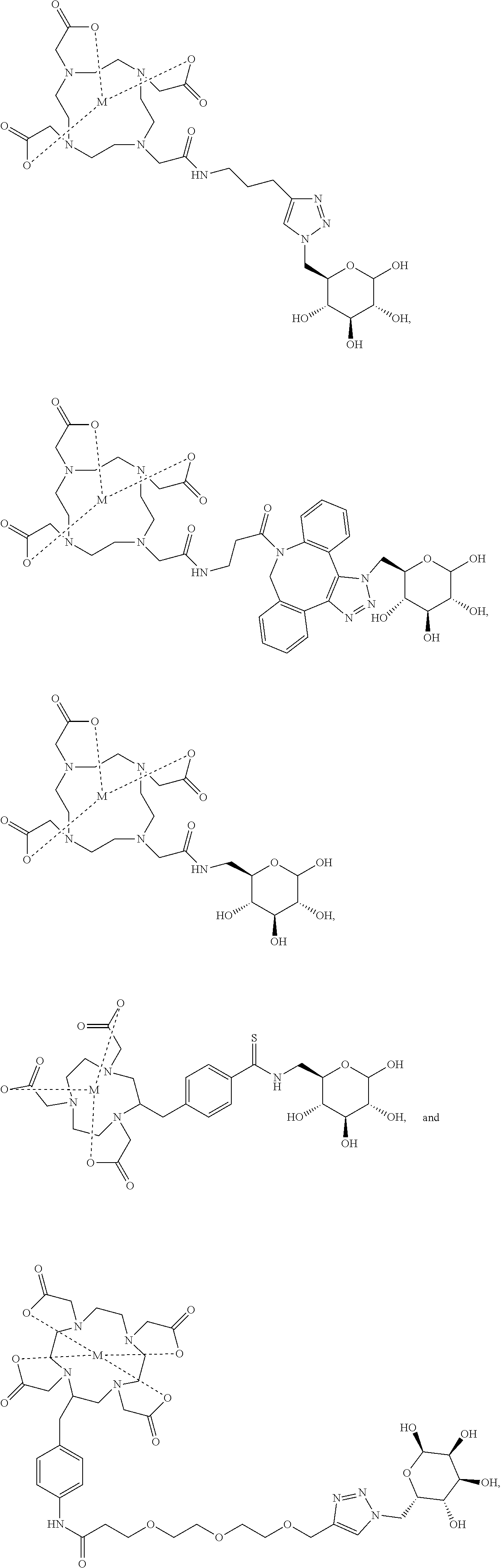
wherein M is a radioisotope.

14. The compound of claim 13, wherein the radioisotope is selected from ⁶⁸Ga, ⁶⁴Cu, ¹⁷⁷Lu, ⁸⁹Zr, ⁸⁶Y, ⁹⁰Y, ⁴³Sc, ⁴⁴Sc, ¹⁵²Tb, ⁸²Rb, ²²⁵Ac, ²¹¹At, ²²⁷Th, ²¹³Bi, ²²⁴Ra, ²²³Ra, ⁸⁹Sr, and ¹⁵³Sm.

15. A method for the treatment of a disorder in a subject, the method comprising administering to the subject an effective amount of at least one compound of claim 1, wherein the disorder is a disorder of uncontrolled cellular proliferation, a neurological disorder, atherosclerosis, diabetes, or heart disease.

16. The method of claim 15, wherein the disorder is a cancer is selected from a sarcoma, a carcinoma, a hematological cancer, a solid tumor, breast cancer, cervical cancer, gastrointestinal cancer, colorectal cancer, brain cancer, skin cancer, prostate cancer, ovarian cancer, thyroid cancer, testicular cancer, pancreatic cancer, endometrial cancer, melanoma, glioma, leukemia, lymphoma, chronic myeloproliferative disorder, myelodysplastic syndrome, myeloproliferative neoplasm, and plasma cell neoplasm (myeloma).

17. A method for modifying sodium-dependent glucose transporter (SGLT) expression in a subject, the method comprising administering to the subject an effective amount of at least one compound of claim 1.

18. The method of claim 17, wherein modifying is inhibiting.

19. The method of claim 17, wherein modifying is via knock-down or knock-up of SGLT expression.

20. The method of claim 17, wherein the disorder associated with SGLT expression is a disorder of uncontrolled cellular proliferation, diabetes, heart disease, or a neurological disorder.
